(19) United States
(12) Patent Application Publication
(10) Pub. No.: US 2025/0354198 A1
(43) Pub. Date: Nov. 20, 2025

(54) METHODS FOR DETECTING AND ISOLATING EXTRACELLULAR VESICLES

(71) Applicant: MESO SCALE TECHNOLOGIES, LLC., Rockville, MD (US)

(72) Inventors: Evan GIZZIE, Washington, DC (US); David ROUTENBERG, Gaithersburg, MD (US); Alexander K. TUCKER-SCHWARTZ, Bethesda, MD (US)

(21) Appl. No.: 18/860,884

(22) PCT Filed: Apr. 28, 2023
(86) PCT No.: PCT/US2023/020399

§ 371 Date: Oct. 28, 2024

Related U.S. Application Data
(60) Provisional application No. 63/336,608, filed on Apr. 29, 2022.
(60) Provisional application No. 63/336,606, filed on Apr. 29, 2022.

(51) Int. Cl. C12Q 1/6816; C12Q 1/6855; C12Q 1/6869; C12Q 1/6876

(52) U.S. Cl. CPC C12Q 1/6816; C12Q 1/6855; C12Q 1/6869; C12Q 1/6876

(57) ABSTRACT

The disclosure relates to method and kits for highly specific detection and quantification of extracellular vesicles (EVs) by targeting at least two EV surface markers using binding reagents. The binding reagents contain hybridisation sequences and primer binding regions, the hybridisation sequences are ligated and amplified using primers. The disclosure further relates to methods and kits for isolating EVs using capture entities, splint entities and staple entities wherein the capture entity comprises a labile linkage.

### GOVERNMENT LICENSE RIGHTS

This invention was made with government support under grant number MH118167 awarded by the National Institutes of Health and grant number TR002886 awarded by the National Institutes of Health. The government has certain rights in the invention.

### SEQUENCE LISTING

The instant application includes a Sequence Listing which has been submitted electronically in XML ST26 format and is hereby incorporated by reference in its entirety. The Sequence Listing, created on Jan. 15, 2025, is named 0076-0004EP5.xml and is 10,283 bytes in size.

### FIELD OF THE DISCLOSURE

The disclosure relates to methods and kits for highly specific detection, isolation and analysis of extracellular vesicles (EVs) by targeting at least two EV surface markers.

### BACKGROUND

Extracellular vesicles (EVs) are a diverse group of cell-secreted membrane vesicles implicated in a wide variety of physiological and pathological processes, many of which are only beginning to be understood. These include immune regulation, antigen presentation, tumor progression and metastasis, modulation of inflammation, stem cell regulation, neuronal development and regeneration, and cell-to-cell transfer of pathogenic proteins and nucleic acids. EVs are secreted from nearly all cell types through multiple mechanisms including the fusion of specific endosomal compartments called multivesicular bodies (MVB) with the plasma membrane and by budding/shedding directly from the plasma membrane. EVs are present in nearly all body fluids including blood, urine, cerebral spinal fluid, and saliva, and are secreted by most in vitro cultured cells as well. Because of the EV formation mechanisms, EVs contain specific lipids, membrane proteins, and internalized proteins, nucleic acids and metabolites derived from their cells of origin and are thus a rich source of potential biomarkers.

Recent research suggests a role for EVs in the function of the healthy central nervous system (CNS) as well as a role in numerous diseases of the CNS. Many cells of the CNS including neurons, astrocytes, oligodendroglia and microglia have been shown to secrete EVs in vitro. In neurons, synaptic activity-dependent EV release and reuptake has been observed and has been proposed as a possible mechanism of synaptic plasticity and inter-neuronal transfer of complex information. Neurons have also been shown to transfer miRNA via EVs to astrocytes, modulating the level of an important functional synaptic protein, EAAT2/GLT1. EV secretion by oligodendrocytes has been found to modulate myelin biogenesis, promote neuronal viability under stress and enable degradation of oligodendroglial membrane proteins by a subset of microglia through an “immunologically silent” macropinocytotic mechanism. Astrocyte-derived EVs have been shown to promote neuronal survival under stress by transferring heat-shock proteins and synapsin I. EV secretion by microglia has been shown to be inducible by Wnt-signaling and to stimulate synaptic activity by enhancing sphingosine metabolism in neurons and to represent a unique secretion mechanism for IL-1beta, an important neuroinflammatory cytokine.

In addition to promoting healthy CNS function, EVs appear to play several roles in various CNS diseases and disorders. Broadly these include the export of toxic proteins and possibly promotion of toxic isoform formation, mediation of neuroinflammation, and the transfer of disease associated miRNAs. Numerous studies have demonstrated that EVs can mediate the transfer of toxic proteins between cells both in-vitro and in animal studies. This includes the misfolded prion protein PrPSc, the infectious agent in human diseases Creutzfeldt-Jakob disease (CJD) and Gerstmann-Sträussler-Scheinker syndrome (GSS), aggregated alpha-synuclein, the pathogenic species associated with Parkinson's disease and Lewy body dementia, aggregated Tau and beta-amyloid peptides hallmarks of Alzheimer's disease (AD), frontotemporal lobar degeneration (FTD) and progressive supranuclear palsy (PSP), and mutated SOD1, linked to the development of amyotrophic lateral sclerosis (ALS). There is also some evidence that the secretion of toxic proteins in EVs may actually have a protective role, facilitating clearing of these pathogenic species by microglia.

Assessing the composition of EVs generally requires isolating a pure population of EVs and separating it from non-EV associated factors. Some demonstrations of this idea have focused on enriching CNS-derived extracellular vesicles (CNS-EVs) from plasma or serum based on immunoaffinity capture of specific EV surface proteins and measuring disease-associated proteins within the enriched EV population.

Despite its utility, this method has significant fundamental and technical drawbacks. Fundamentally, the use of a single marker for EV isolation presents a great challenge. Most surface proteins are expressed on a variety of cell types; thus, multiple markers are usually needed to define a specific cell population. This is often apparent in flow cytometry, wherein multiple markers are usually employed despite the benefit of a predefined input cell population (e.g. PBMCs, or cultured cells). When isolating EVs from blood, nearly all cell types of the organism may be represented within the EV population, increasing the challenge of identifying a single marker specific for EVs from one cell type. Technical challenges of the existing approach are illustrated by the dramatic differences in levels of circulating LICAM+EVs and associated cargo molecules (e.g. Tau) reported by multiple groups using nearly identical protocols. This variability, which likely stems from minor variations in protocols from lab to lab (e.g. wash or mixing steps) speaks to the need for protocol standardization and simplification.

Multimarker isolation of EVs using stapling involves (i) immobilizing an EV of interest by binding it to a surface through a reversible linkage by, for example, binding a first surface protein on the EV to a capture entity immobilized on the surface by a cleavable linker, (ii) further binding or “stapling” an immobilized EV to the surface through one or more additional linkages that target one or more additional distinct features of the EV, typically through binding an oligonucleotide-conjugated entity to a second surface protein on the EV and connecting the oligonucleotide to a second oligonucleotide associated with the surface through e.g. hybridization or enzymatic ligation, and (iii) breaking the first linkage between the EVs and surface to release any EVs that were not also bound to the surface through the additional linkage(s) or “stapled” to the surface, thereby retaining only the captured EVs having the features targeted by the additional linkage(s).

See, for example, PCT application publication number WO2019222708, which is hereby incorporated by reference in its entirety. The surface may be, but is not limited to, a particle, a bead, or a surface of a culture dish, culture well, or plate. The surface may be magnetic or it may be coated with an electrode.

Such methods permit isolation and/or enrichment of specific populations of EVs or other surface marker displaying agents of interest with specific features e.g. EV with combinations of two or more surface proteins.

After capture of EVs, “stapling”, and removal of non-target EVs by breaking the first linkage (for example, via washing the surface in a stringent wash buffer to dissociated the strands of a duplex DNA linker, or through cutting a double stranded DNA linker using a sequence-specific endonuclease, or through a chemically cleavable linker), final release of the desired EV population is typically performed by breaking the attachment between the surface and additional linkages, e.g. by using a non-specific endonuclease such as DNAse I to digest oligonucleotides where the additional linkages are provide by oligonucleotide-conjugated antibodies. This leaves antibodies bound to EVs through antibody: antigen interaction which can interfere with down downstream analysis of the selected population.

There has been growing interest in measuring EVs as biomarkers for characterizing biological processes in cell lines or model organisms, or as diagnostic indicators of disease processes in people. To support the growing field of EV research, there is a need for sensitive, accurate and reproducible assays to accurately characterize and quantify EVs. Since it is the surface EV protein composition that will largely dictate their biological behavior, high-throughput single EV profiling methods are needed to better define EV subpopulations. Current immunoassays for measuring intact EVs are based on the presence of one or more EV surface proteins. These methods enable quantitative and qualitative comparisons of the number and character of EVs in complex biological samples.

One existing method for screening EVs is a combinatorial screening method based on a proximity extension ligation (PEL) reactions that only produce signal (amplifiable DNA with 3 barcodes) when three antibodies are bound to the same EV. See, e.g., PCT patent application publication nos. WO2020086751 and WO2022051481, incorporated by reference in their entirety. While this reaction has high specificity, it would be desirable to increase the efficiency of converting antibody triplets into full length DNA, thus reducing bottlenecks for sequencing analysis.

### SUMMARY OF THE DISCLOSURE

The present disclosure provides a method of detecting an extracellular vesicle (EV) of interest in a sample, comprising: a. contacting the sample with: (i) a capture reagent bound to a surface; (ii) a first binding reagent that binds a first surface marker of the EV, wherein the first binding reagent comprises a first detection sequence that comprises a first hybridization sequence, and a first amplification primer site; (iii) a second binding reagent that binds a second surface marker of the EV, wherein the second binding reagent comprises a second detection sequence that comprises a second hybridization sequence, a third hybridization sequence, and a fourth hybridization sequence; (iv) a third binding reagent comprising a third detection sequence, wherein the third detection sequence comprises a fifth hybridization sequence, and a second amplification primer site; and (v) an oligonucleotide insert comprising an oligonucleotide insert sequence; wherein the first hybridization sequence and the second hybridization sequence are complementary; wherein the fourth hybridization sequence and the fifth hybridization sequence are complementary; and wherein the third hybridization sequence is complementary to the oligonucleotide insert sequence; b. forming a single output oligonucleotide comprising: (i) ligating the hybridized first detection sequence to the hybridized oligonucleotide insert; and (ii) ligating the hybridized oligonucleotide insert to the third detection sequence; and c. amplifying the single output oligonucleotide using a first primer that hybridizes to the first amplification primer site and a second primer that hybridizes to the second amplification primer site. In embodiments, the method employs multiple different capture reagents and multiple different first, second, and third binding reagents to allow combinatorial analysis of markers. In embodiments, the capture reagent is releasably bound to the surface.

In embodiments, the third binding reagent binds to a third surface marker and the capture reagent bound to a surface (i.e., immobilized) binds to a known EV surface marker such as CD9, CD63, CD81, or another tetraspanin associated with EVs. The capture reagent may be immobilized on a surface such as a bead. The immobilized capture reagent captures EVs having the known surface marker targeted by the capture reagent, and the other surface markers can be identified by determining the sequence of the output oligonucleotide.

In embodiments, the third binding reagent comprising the third detection sequence is bound to the same surface as the capture reagent. Accordingly, a method of detecting an EV of interest in a sample according to this embodiment comprises: a. contacting the sample with: (i) a capture reagent bound to a surface; (ii) a first binding reagent that binds a first surface marker of the EV, wherein the first binding reagent comprises a first detection sequence that comprises a first hybridization sequence, and a first amplification primer site; (iii) a second binding reagent that binds a second surface marker of the EV, wherein the second binding reagent comprises a second detection sequence that comprises a second hybridization sequence, a third hybridization sequence, and a fourth hybridization sequence; (iv) a third binding reagent that comprises a third detection sequence and that is bound to the same surface as the capture reagent, wherein the third detection sequence comprises a fifth hybridization sequence, and a second amplification primer site; and (v) an oligonucleotide insert comprising an oligonucleotide insert sequence; wherein the first hybridization sequence and the second hybridization sequence are complementary; wherein the fourth hybridization sequence and the fifth hybridization sequence are complementary; and wherein the third hybridization sequence is complementary to the oligonucleotide insert sequence; b. forming a single output oligonucleotide comprising: (i) ligating the hybridized first detection sequence to the hybridized oligonucleotide insert; and (ii) ligating the hybridized oligonucleotide insert to the third detection sequence; and c. amplifying the single output oligonucleotide using a first primer that hybridizes to the first amplification primer site and a second primer that hybridizes to the second amplification primer site. This method is particularly useful for detecting EVs or other surface marker displaying agents (SMDAs) that may not harbor a known tetraspanin (or other known protein) on their surface. In such a situation, a variety of beads, each having a different capture reagent and corresponding third binding reagent, may be used to determine which capture reagent binds an EV in a sample (thus indicating that the captured EV harbors the surface marker associated with the bead). By sequencing the resulting output oligonucleotide, the combination of surface markers associated with the captured EV can be elucidated.

The present disclosure also provides a method of determining surface markers of an SMDA, a method of identifying SMDAs that harbor combinations of surface markers, a method of detecting populations of SMDAs having certain surface markers, and/or a method of detecting or quantifying multiple populations of SMDAs where each population has a specific set of surface markers, the method comprising contacting the SMDA with a capture reagent bound to a surface, a plurality of unique binding reagents, and an oligonucleotide insert, wherein each unique binding reagent comprises a detection sequence comprising a unique barcode oligonucleotide sequence, wherein when at least three unique binding reagents bind to three unique surface markers of the SMDA, an output oligonucleotide is generated that comprises the barcode oligonucleotide sequences of each of the three unique binding reagents, wherein the output oligonucleotide is capable of being sequenced to identify the three unique surface markers of the SMDA, and further wherein the plurality of binding reagents comprises: a. a first binding reagent comprising a first detection sequence that comprises a first hybridization sequence, and a first amplification primer site; b. a second binding reagent comprising a second detection sequence that comprises a second hybridization sequence, a third hybridization sequence, and a fourth hybridization sequence; and c. a third binding reagent comprising a third detection sequence that comprises a fifth hybridization sequence, and a second amplification primer site, wherein the first hybridization sequence and the second hybridization sequence are complementary; wherein the fourth hybridization sequence and the fifth hybridization sequence are complementary; wherein the third hybridization sequence is complementary to the oligonucleotide insert sequence; and wherein generating the single output oligonucleotide comprises ligating the hybridized first detection sequence to the hybridized oligonucleotide insert and ligating the hybridized oligonucleotide insert to the third detection sequence. The SMDA may be, for example, an EV. In embodiments, the method employs multiple capture reagents and multiple first, second, and third binding reagents to allow combinatorial analysis of markers. In embodiments of this method, the third binding reagent binds to a third surface marker and the capture reagent bound to a surface (i.e., immobilized) binds to a known EV surface marker such as CD9, CD63, CD81, or another tetraspanin associated with EVs. The capture reagent may be immobilized on a surface such as a bead. The immobilized capture reagent captures EVs having the known surface marker targeted by the capture reagent, and the other surface markers can be identified by determining the sequence of the output oligonucleotide.

In embodiments of this method, the capture reagent and the third binding reagent are bound to the same surface. Accordingly, a method of determining surface markers of an SMDA, a method of identifying SMDAs that harbor combinations of surface markers, a method of detecting populations of SMDAs having certain surface markers, and/or a method of detecting or quantifying multiple populations of SMDAs where each population has a specific set of surface markers according to this embodiment comprises: a. contacting an SMDA with: (i) a capture reagent bound to a surface; (ii) a first binding reagent that binds a first surface marker of the SMDA, wherein the first binding reagent comprises a first detection sequence that comprises a first hybridization sequence, and a first amplification primer site; (iii) a second binding reagent that binds a second surface marker of the SDMA, wherein the second binding reagent comprises a second detection sequence that comprises a second hybridization sequence, a third hybridization sequence, and a fourth hybridization sequence; (iv) a third binding reagent that comprises a third detection sequence and that is bound to the same surface as the capture reagent, wherein the third detection sequence comprises a fifth hybridization sequence, and a second amplification primer site; and (v) an oligonucleotide insert comprising an oligonucleotide insert sequence; wherein the first hybridization sequence and the second hybridization sequence are complementary; wherein the fourth hybridization sequence and the fifth hybridization sequence are complementary; and wherein the third hybridization sequence is complementary to the oligonucleotide insert sequence; b. forming a single output oligonucleotide comprising: (i) ligating the hybridized first detection sequence to the hybridized oligonucleotide insert; and (ii) ligating the hybridized oligonucleotide insert to the third detection sequence; and c. amplifying the single output oligonucleotide using a first primer that hybridizes to the first amplification primer site and a second primer that hybridizes to the second amplification primer site. This method is particularly useful for detecting EVs or other SMDAs that may not harbor a known tetraspanin (or other known protein) on their surface. In such a situation, a variety of beads, each having a different capture reagent and corresponding third binding reagent, may be used to determine which capture reagent binds an SMDA in a sample (thus indicating that the captured SMDA harbors the surface marker associated with the bead). By sequencing the resulting output oligonucleotide, the combination of surface markers associated with the captured SDMA can be elucidated.

The present disclosure also provides a method of isolating multimarker extracellular vesicles (EVs) from a sample, comprising contacting a sample suspected of containing a multimarker EV with a first oligonucleotide-conjugated capture entity, a second oligonucleotide-conjugated splint entity, a third oligonucleotide-conjugated staple entity, and a surface; wherein each oligonucleotide-conjugated entity is specific for a different EV surface marker; wherein the first oligonucleotide-conjugated capture entity comprises a first oligonucleotide comprising a first Target nucleotide sequence, and a uracil-DNA glycosylase 1 (UDG1) labile linkage sequence, wherein the capture entity is conjugated to the first oligonucleotide so that the first Target nucleotide sequence is located between the capture entity and the UDG1 labile linkage sequence; wherein the third oligonucleotide-conjugated staple entity comprises a third oligonucleotide comprising a third Target nucleotide sequence and a restriction enzyme cleavage site, wherein the staple entity is conjugated to the third oligonucleotide so that the third Target nucleotide sequence is located between the staple entity and the restriction enzyme cleavage site; wherein the second oligonucleotide-conjugated splint entity comprises a second oligonucleotide comprising a second Target nucleotide sequence, a restriction enzyme cleavage site and an additional nucleic acid sequence, wherein the splint entity is conjugated to the second oligonucleotide so that the second Target nucleotide sequence is located between the splint entity and the restriction enzyme cleavage site and the additional nucleic acid sequence is located on the side of the second Target nucleotide sequence (either upstream or downstream) opposite the side that is conjugated to the splint entity; wherein complementary DNA sequences on the second oligonucleotide-conjugated splint entity and the third oligonucleotide-conjugated staple entity hybridize to form a double-stranded DNA restriction site, and wherein the surface has two capture oligonucleotides immobilized thereon, wherein the first capture oligonucleotide comprises a sequence that is complementary to the additional nucleic acid sequence of the second oligonucleotide of the second oligonucleotide-conjugated splint entity, and is capable of ligating to the end of the third oligonucleotide away from the third oligonucleotide-conjugated staple entity; and the second capture oligonucleotide comprises a sequence that is complementary to the UDG1 labile linkage sequence of the first oligonucleotide on the first oligonucleotide-conjugated capture entity.

In embodiments of the method of isolating EVs, the capture entity, splint entity and staple entity are each independently selected from an antibody or antigen binding fragment thereof, antigen, ligand, receptor, oligonucleotide, hapten, epitope, mimitope, lipid binding protein, carbohydrate binding protein, DNA aptamer or RNA aptamer. In embodiments, the capture entity, splint entity and staple entity are each an antibody or an antibody fragment.

In embodiments of the method of isolating EVs, the Target nucleotide sequences remain intact following EV release and can be hybridized to complementary labeled probes (complementary to the Target sequence). In embodiments, the probes allow the EV to be fluorescently labeled, for example, labeled for multi-color co-localization microscopy/FISH microscopy. In embodiments, the probes are dye-conjugated in situ probes. In embodiments, a different label, e.g., fluorescent label, is attached to each probe directed to a different Target sequence. In other embodiments, one or more of the Target sequences is hybridized to a complementary probe that is capable of subsequent EV pulldown or recapture onto a streptavidin surface for additional analyses including, e.g., electrochemiluminescent assays. In embodiments, the complementary probe is biotinylated and the surface comprises avidin or streptavidin.

The present disclosure also provides a construct for isolating multimarker extracellular vesicles (EVs) comprising: a first oligonucleotide-conjugated capture entity, a second oligonucleotide-conjugated splint entity, a third oligonucleotide-conjugated staple entity, and a surface; wherein each oligonucleotide-conjugated entity is specific for a different EV surface marker; wherein the first oligonucleotide-conjugated capture entity comprises a first oligonucleotide comprising a first Target nucleotide sequence, and a UDG1 labile linkage sequence, wherein the capture entity is conjugated to the first oligonucleotide so that the first Target nucleotide sequence is located between the capture entity and the UDG1 labile linkage sequence; wherein the third oligonucleotide-conjugated staple entity comprises a third oligonucleotide comprising a third Target nucleotide sequence and a restriction enzyme cleavage site, wherein the staple entity is conjugated to the third oligonucleotide so that the third Target nucleotide sequence is located between the staple entity and the restriction enzyme cleavage site; wherein the second oligonucleotide-conjugated splint entity comprises a second oligonucleotide comprising a second Target nucleotide sequence, a restriction enzyme cleavage site and an additional nucleic acid sequence, wherein the splint entity is conjugated to the second oligonucleotide so that the second Target nucleotide sequence is located between the splint entity and the restriction enzyme cleavage site and the additional nucleic acid sequence is located on the side of the second Target nucleotide sequence (either upstream or downstream) opposite the side that is conjugated to the splint entity; wherein complementary DNA sequences on the second oligonucleotide-conjugated splint entity and the third oligonucleotide-conjugated staple entity hybridize to form a double-stranded DNA restriction site, and wherein the surface has two capture oligonucleotides immobilized thereon, wherein the first capture oligonucleotide comprises a sequence that is complementary to the additional nucleic acid sequence of the second oligonucleotide of the second oligonucleotide-conjugated splint entity, and is capable of ligating to the end of the third oligonucleotide away from the third oligonucleotide-conjugated staple entity; and the second capture oligonucleotide comprises a sequence that is complementary to the UDG1 labile linkage sequence of the first oligonucleotide on the first oligonucleotide-conjugated capture entity.

In embodiments of the construct for isolating EVs, the capture entity, splint entity and staple entity are each independently selected from an antibody or antigen binding fragment thereof, antigen, ligand, receptor, oligonucleotide, hapten, epitope, mimitope, lipid binding protein, carbohydrate binding protein, DNA aptamer or RNA aptamer. In embodiments, the capture entity, splint entity and staple entity are each an antibody or an antibody fragment.

The present disclosure further provides a kit for detecting an EV in a sample comprising, in one or more vials, containers, or compartments: (i) a capture reagent; (ii) a first binding reagent that binds a first surface marker of the EV, wherein the first binding reagent comprises a first detection sequence that comprises a first hybridization sequence, and a first amplification primer site; (iii) a second binding reagent that binds a second surface marker of the EV, wherein the second binding reagent comprises a second detection sequence that comprises a second hybridization sequence, a third hybridization sequence, and a fourth hybridization sequence; (iv) a third binding reagent comprising a third detection sequence wherein the third detection sequence comprises a fifth hybridization sequence and a second amplification primer site; and (v) an oligonucleotide insert comprising an oligonucleotide insert sequence; wherein the first hybridization sequence and the second hybridization sequence are complementary; wherein the fourth hybridization sequence and the fifth hybridization sequence are complementary; and wherein the third hybridization sequence is complementary to the oligonucleotide insert sequence.

In embodiments, the capture reagent is bound to a surface. In embodiments, the third binding reagent is bound to the same surface as the capture reagent. In embodiments, the surface is a bead. In embodiments, the surface is a plate bottom.

In embodiments, the third binding reagent binds a third surface marker of the EV.

In embodiments, the kit further comprises a first primer complementary to the first amplification primer site and a second primer complementary to the second amplification primer site. In embodiments, the kit comprises multiple different capture reagents and multiple different first, second, and third binding reagents to allow combinatorial analysis of markers.

The present disclosure further provides a kit for determining surface markers of an SMDA, for identifying SMDAs that harbor combinations of surface markers, for detecting populations of SMDAs having certain surface markers, and/or for detecting or quantifying multiple populations of SMDAs where each population has a specific set of surface markers, the kit comprising, in one or more vials, containers, or compartments: a capture reagent, at least three unique binding reagents, and an oligonucleotide insert, wherein each unique binding reagent comprises a detection sequence comprising a unique barcode oligonucleotide sequence, wherein when at least three unique binding reagents bind to three unique surface markers of the surface marker displaying agent, an output oligonucleotide is generated that comprises the barcode oligonucleotide sequences of each of the three unique binding reagents, wherein the output oligonucleotide is capable of being sequenced to identify the three unique surface markers of the surface marker displaying agent, and further wherein the plurality of binding reagents comprises: a. a first binding reagent comprising a first detection sequence that comprises a first hybridization sequence, and a first amplification primer site; b. a second binding reagent comprising a second detection sequence that comprises a second hybridization sequence, a third hybridization sequence, and a fourth hybridization sequence; and c. a third binding reagent comprising a third detection sequence that comprises a fifth hybridization sequence, and a second amplification primer site, wherein the first hybridization sequence and the second hybridization sequence are complementary; wherein the fourth hybridization sequence and the fifth hybridization sequence are complementary; wherein the third hybridization sequence is complementary to the oligonucleotide insert sequence.

In embodiments, the capture reagent is bound to a surface. In embodiments, the third binding reagent is bound to the same surface as the capture reagent. In embodiments, the surface is a bead. In embodiments, the surface is a plate bottom.

In embodiments, the third binding reagent binds a third surface marker of the EV.

In embodiments, the kit further comprises a first primer complementary to the first amplification primer site and a second primer complementary to the second amplification primer site. In embodiments, the kit comprises multiple different capture reagents and multiple different first, second, and third binding reagents to allow combinatorial analysis of markers.

The present disclosure further provides a kit for isolating multimarker extracellular vesicles (EVs) comprising, in one or more vials, containers, or compartments: a first oligonucleotide-conjugated capture entity, a second oligonucleotide-conjugated splint entity, a third oligonucleotide-conjugated staple entity, and a surface; wherein each oligonucleotide-conjugated entity is specific for a different EV surface marker; wherein the first oligonucleotide-conjugated capture entity comprises a first oligonucleotide comprising a first Target nucleotide sequence, and a UDG1 labile linkage sequence, wherein the capture entity is conjugated to the first oligonucleotide so that the first Target nucleotide sequence is located between the capture entity and the UDG1 labile linkage sequence; wherein the third oligonucleotide-conjugated staple entity comprises a third oligonucleotide comprising a third Target nucleotide sequence and a restriction enzyme cleavage site, wherein the staple entity is conjugated to the third oligonucleotide so that the third Target nucleotide sequence is located between the staple entity and the restriction enzyme cleavage site; wherein the second oligonucleotide-conjugated splint entity comprises a second oligonucleotide comprising a second Target nucleotide sequence, a restriction enzyme cleavage site and an additional nucleic acid sequence, wherein the splint entity is conjugated to the second oligonucleotide so that the second Target nucleotide sequence is located between the splint entity and the restriction enzyme cleavage site and the additional nucleic acid sequence is located on the side of the second Target nucleotide sequence away from the splint entity; wherein complementary DNA sequences on the second oligonucleotide-conjugated splint entity and the third oligonucleotide-conjugated staple entity hybridize to form a double-stranded DNA restriction site, and wherein the surface has two capture oligonucleotides immobilized thereon, wherein the first capture oligonucleotide comprises a sequence that is complementary to the additional nucleic acid sequence of the second oligonucleotide of the second oligonucleotide-conjugated splint entity, and is capable of ligating to the end of the third oligonucleotide away from the third oligonucleotide-conjugated staple entity; and the second capture oligonucleotide comprises a sequence that is complementary to the UDG1 labile linkage sequence of the first oligonucleotide on the first oligonucleotide-conjugated capture entity.

In embodiments of the kit for isolating EVs, the capture entity, splint entity and staple entity are each independently selected from an antibody or antigen binding fragment thereof, antigen, ligand, receptor, oligonucleotide, hapten, epitope, mimitope, lipid binding protein, carbohydrate binding protein, DNA aptamer or RNA aptamer. In embodiments, the capture entity, splint entity and staple entity are each an antibody or an antibody fragment.

### BRIEF DESCRIPTION OF THE DRAWINGS

The following drawings form part of the present specification and are included to further demonstrate certain aspects of the present disclosure.

FIG. 1A and FIG. 1B show a proximity ligation-ligation (PLL) method as described in Example 1. FIG. 1A shows a schematic of constructs that can be used in a PLL assay which is shown schematically in FIG. 1B. As shown in FIG. 1B panel v., the final extension product produced when all three EV surface markers are present includes the sequences corresponding to those from the oligonucleotides on the 5-prime, splint and 3-prime oligonucleotide-conjugated antibodies shown in FIG. 1A. F′ and R′ refer to forward and reverse primer sequences, respectively. BC refers to Barcode sequences and H1 and H2 represent the sequences formed by the overlap of the two common overlap sequences with each other as designated in FIG. 1A.

FIG. 2A is a close up schematic of the hybridization of the oligonucleotides in an embodiment of the PLL assay described in Example 1. FIG. 2B shows results comparing the proximity extension ligation (PEL) assay with the PLL assay as described in the Example.

FIG. 3A shows quantitative PCR (qPCR) results of further experiment showing the specificity of the PLL assay as described in Example 1. FIG. 3B shows a melting curve plot for a PLL assay where the 5-prime construct, splint construct and 3-prime construct all target CD81 (plain line; CD81-CD81-CD81) compared to a PLL assay where the splint construct is missing (diamonds; CD81-None-CD81).

FIG. 4A is a schematic of a tetraspanin-independent PLL method where the 3′ barcode oligonucleotide is attached directly to a surface as described in Example 2. FIG. 4B shows qPCR results of a PLL assay using the tetraspanin-independent PLL construct embodied in FIG. 4A.

FIG. 5A is a general schematic showing an embodiment of a multimarker EV antibody-oligonucleotide construct for use in isolating EVs as described in Example 3. FIG. 5B is a detailed schematic showing example oligonucleotide sequences for each conjugate. In this specific embodiment, the capture entity is a capture antibody, the splint entity is a splint antibody and the staple entity is a staple antibody.

### DETAILED DESCRIPTION OF THE DISCLOSURE

### I. Overview

A variety of analytical methods have been used to characterize EVs including, most commonly, immunoassays (Western blotting, flow cytometry, sandwich immunoassays), electron microscopy, mass spectrometry, PCR and sequencing, and nanoparticle tracking. One of the most significant limitations to characterizing EVs has been the difficulty of separating EVs from the other components in complex biofluids.

EV isolation, enrichment, and purification have been the subject of extensive discussion and publication yet there is still not one universally-accepted method. Ultracentrifugation, ultrafiltration, size-exclusion chromatography, and immuno-affinity based methods all have their strengths and shortcomings. Each must be applied in the appropriate situation with full recognition of the potential for introducing bias or allowing contamination by non-EV components of the sample. Analytical methods that avoid pre-purification steps are advantageous as they introduce no bias in the EV population subject to analysis; however, they have the highest risk of negative effects due to non-EV related molecular interactions and artifacts.

The inventors have discovered a surprisingly effective and highly specific method of detecting and isolating EVs of interest from samples. In embodiments, and by way of example, the method indirectly attaches an EV to a surface using at least two, and, in some cases, at least three, separate EV surface markers. In embodiments, the method provides a highly sensitive method of detecting and isolating EVs having a specific combination of multiple surface markers.

In embodiments, the methods described herein for detecting an EV are used in methods of isolating an EV. In embodiments, the kits described herein for use in detecting an EV can be used for isolating an EV.

In embodiments, the methods described herein for isolating an EV are used in detecting an EV. In embodiments, the constructs described herein for use in isolating an EV can be used for detecting an EV. In embodiments, the kits described herein for use in isolating an EV can be used for detecting an EV.

In embodiments, any method described herein for use in detecting an EV are used for detecting a surface marker displaying agent. In embodiments, any method described herein for use in isolating an EV can be used for isolating a surface marker displaying agent (SMDA). SMDAs can be naturally-occurring, partially synthetic, or fully synthetic. In embodiments, an SMDA is a biologically relevant material or component. In general, an SMDA comprises a surface, typically a lipid bilayer, membrane, cell wall, or envelope, on which one or more markers are displayed. In embodiments, the SMDA encapsulates components such as, e.g., proteins, nucleic acids, lipids, carbohydrates, small molecules such as hormones, cofactors, vitamins, minerals, salts, metals, metal-containing compounds, or combination thereof. Examples of SMDAs include cells (including prokaryotic cells such as bacterial cells or archaeal cells; eukaryotic cells such as mammalian cells, insect cells, or plant cells); viruses and viral particles; cellular organelles such as nucleus, endoplasmic reticulum, Golgi apparatus, mitochondria, vacuoles, or chloroplast; vesicles such as lysosome, endosome, peroxisome, and liposome; and extracellular vesicles (EVs) or exosomes. Although the present specification may refer to EVs in certain embodiments, the disclosure contemplates that such aspects also apply to any SMDA provided herein without limitation.

### II. Methods

### Proximity Ligase-Ligase (Two Ligation) Methods

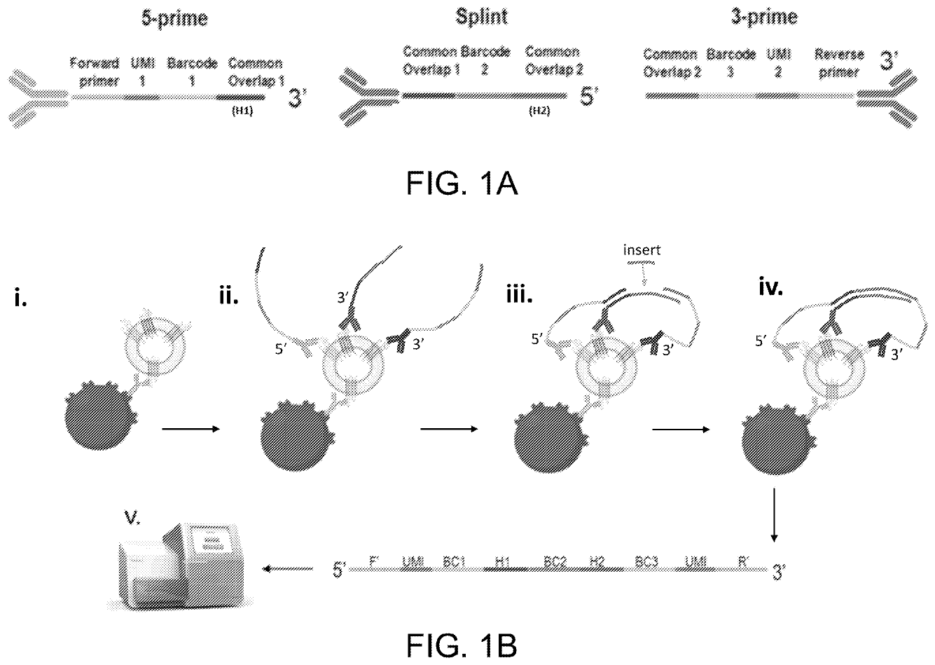

There are limitations to proximity extension ligation methods in certain assays. The present disclosure provides an alternative assay format using two-site ligation (Proximity Ligation Ligation; PLL), embodiments of which are shown in FIGS. 1A. 1B, 2A and 2B. In embodiments, as demonstrated further in the Examples below, this reaction is at least about 50-fold to about 100-fold more efficient than the PEL reaction at converting oligonucleotides on antibody triplets into full-length product. In embodiments, the PLL reaction is at least about 50-fold more efficient than the PEL reaction. In embodiments, the PLL reaction is at least about 100-fold more efficient than the PEL reaction. It also allows for reduced length of the hybridization regions from 10 bases to as low as 5 such that these are only transient interactions, which, in turn, allows elimination of the blocker oligos needed to prevent hybridization and aggregation of unbound conjugates. The PLL reaction shows a reduction in non-specific background over PEL, which is important as the number of antibodies in the pool is scaled to much higher numbers. Overall, it is a simpler, more efficient, more specific system than PEL. This change in the assay may also enable homogenous assays.

In embodiments, provided herein is a method of detecting an extracellular vesicle (EV) of interest in a sample, comprising:
contacting the sample with:
   (i) a capture reagent bound to a surface;
   (ii) a first binding reagent that binds a first surface marker of the EV, wherein the first binding reagent comprises a first detection sequence that comprises a first hybridization sequence, and a first amplification primer site;
   (iii) a second binding reagent that binds a second surface marker of the EV, wherein the second binding reagent comprises a second detection sequence that comprises a second hybridization sequence, a third hybridization sequence, and a fourth hybridization sequence;
   (iv) a third binding reagent comprising a third detection sequence bound to a surface, wherein the third detection sequence comprises a fifth hybridization sequence, and a second amplification primer site; and
   (v) an oligonucleotide insert comprising an oligonucleotide insert sequence.

In embodiments of the method, the first hybridization sequence and the second hybridization sequence are complementary. In embodiments, the fourth hybridization sequence and the fifth hybridization sequence are complementary. In embodiments, the third hybridization sequence is complementary to the oligonucleotide insert sequence. In embodiments, the third detection sequence is releasably bound to the surface.

In embodiments, the method employs multiple different capture reagents and multiple different first, second, and third binding reagents to allow combinatorial analysis of markers.

In embodiments, the capture reagent is attached to the surface. In embodiments, the capture reagent is releasably attached to the surface. In embodiments, the capture reagent is non-releasably attached to the surface. In embodiments, the capture reagent is attached to the surface through a binding interaction comprising antibody or antigen binding fragment thereof/antigen or epitope or hapten or mimotope, antigen/antibody or antigen binding fragment thereof, ligand/receptor, receptor/ligand, oligonucleotide/oligonucleotide, hapten/antibody or antigen binding fragment thereof, epitope/antibody or antigen binding fragment thereof, mimitope/antibody or antigen binding fragment thereof, or aptamer/target molecule. In embodiments, the capture reagent is bound to the surface through a streptavidin/biotin or avidin/biotin binding interaction.

In embodiments of the methods of the disclosure, the capture reagent is releasably bound to the surface. In embodiments, the capture reagent is releasably bound to the surface by a labile linker. In embodiments, the labile linker is a heat-labile, a photolabile, or a chemically labile linker. In additional embodiments, the labile linker is an oligonucleotide that is complementary to an oligonucleotide bound to the surface or is an oligonucleotide comprising a restriction site cleavable by a restriction endonuclease. In embodiments, the labile linker is a small molecule that binds to a protein on the surface. In embodiments, the capture reagent is biotinylated, and the surface is coated with streptavidin. The surface can be, for example, a Meso Scale Discovery (MSD) plate electrode or a particle. In some embodiments, the surface is directly coated with the capture reagent. In embodiments, releasing the capture agent from the surface comprises denaturing the labile linker.

In embodiments of the method of detecting, the capture reagent binds to a surface marker common to EVs. Surface markers common to EVs are described herein. In embodiments, the marker is a tetraspanin. In embodiments, the tetraspanin is CD9, CD63, or CD81. In embodiments, the capture reagent binds to a surface marker that is not common to EVs. In embodiments, the capture reagent binds to a surface marker selected from CD2, CD3, CD4, CD5, CD8, CD10 (NEP), CD11b (ITGAM), CD13 (AAP), CD14, CD15 (SSEA-1), CD16 (FcγRIII), CD18 (ITGB2), CD25 (IL-2Ra), CD26 (DPPIV), CD28, CD29 (ITGB1), CD31 (PECAM-1), CD32b (FcγRII), CD33 (Siglec-3), CD36 (GPIV), CD38, CD40, CD41 (GP2B), CD42b (GP1B), CD42a (GP9), CD44 (HCAM), CD45 (LCA), CD50 (ICAM3), CD54 (ICAM-1), CD61 (GP3A), CD62 (P-Selectin), CD62e (E-Selectin), CD62L (L-Selectin), CD64 (FcγRI), CD66a (CEACAM1), CD66e (CEACAM5), CD68 (LAMP4), CD73 (NT5E), CD95 (FAS), CD105 (Endoglin), CD106 (VCAM-1), CD127 (IL-7Ra), CD141 (Thrombomodulin), CD144 (VE-Cadherin), CD146 (MCAM), CD163, CD166 (ALCAM), CD183 (CXCR3), CD204 (MSR1), CD223 (LAG-3), CD309 (VEGFR2), CD324 (E-Cadherin), CD325 (N-Cadherin), CD326 (EpCAM), CD340 (ERBB2), EphA2, CD202B (TIE2), CX3CR1, ITGB5, HLA-A/B/C, HLA-DR/DP/DQ, ESAM, EGFR, FAPa, FLT-1 (VEGFR1) and GLUT1 (SLC2A1).

In embodiments, each of the binding reagents comprises an antibody or antigen binding fragment thereof, antigen, ligand, receptor, oligonucleotide, hapten, epitope, mimitope, lipid binding protein, carbohydrate binding protein, DNA aptamer or RNA aptamer. Thus, in embodiments, the binding reagent/surface marker pairs comprise antibody or antigen binding fragment thereof/antigen or epitope or hapten or mimotope, antigen/antibody or antigen binding fragment thereof, ligand/receptor, receptor/ligand, oligonucleotide/oligonucleotide, hapten/antibody or antigen binding fragment thereof, epitope/antibody or antigen binding fragment thereof, mimitope/antibody or antigen binding fragment thereof, lipid binding protein/target lipid, carbohydrate binding protein/target carbohydrate, or aptamer/target molecule.

In embodiments of the method, the third binding reagent binds to a third surface marker on the EV. In embodiments, the third binding reagent is attached to the same surface as the capture reagent.

In embodiments of the method, each unique binding reagent comprises a detection sequence comprising a unique barcode oligonucleotide sequence. In embodiments, when at least three unique binding reagents bind to three unique surface markers of the surface marker displaying agent, an output oligonucleotide is generated that comprises the barcode oligonucleotide sequences of each of the three unique binding reagents. In embodiments, the output oligonucleotide is capable of being sequenced to identify the three unique surface markers of the surface marker displaying agent.

In embodiments of the method, the plurality of binding reagents comprises:
a. a first binding reagent comprising a first detection sequence that comprises a first hybridization sequence, and a first amplification primer site;
b. a second binding reagent comprising a second detection sequence that comprises a second hybridization sequence, a third hybridization sequence, and a fourth hybridization sequence; and
c. a third binding reagent comprising a third detection sequence that comprises a fifth hybridization sequence, and a second amplification primer site.

In embodiments of the method, the first hybridization sequence and the second hybridization sequence are complementary. In embodiments, the fourth hybridization sequence and the fifth hybridization sequence are complementary. In embodiments, the third hybridization sequence is complementary to the oligonucleotide insert sequence. In embodiments, generating the single output oligonucleotide comprises ligating the hybridized first detection sequence to the hybridized oligonucleotide insert, and ligating the hybridized oligonucleotide insert to the third detection sequence.

In embodiments of the method, each of the binding reagents comprises an antibody or antigen binding fragment thereof, antigen, ligand, receptor, oligonucleotide, hapten, epitope, mimitope, lipid binding protein, carbohydrate binding protein, DNA aptamer or RNA aptamer. Thus, in embodiments, the binding reagent/surface marker pairs comprise antibody or antigen binding fragment thereof/antigen or epitope or hapten or mimotope, antigen/antibody or antigen binding fragment thereof, ligand/receptor, receptor/ligand, oligonucleotide/oligonucleotide, hapten/antibody or antigen binding fragment thereof, epitope/antibody or antigen binding fragment thereof, mimitope/antibody or antigen binding fragment thereof, lipid binding protein/target lipid, carbohydrate binding protein/target carbohydrate, or aptamer/target molecule.

In some discussions of the method, the first binding reagent may also be referred to herein as a “5-prime conjugate,” the second binding reagent may also be referred to as a “splint conjugate,” and the third binding reagent may also be referred to as a “3-prime conjugate.” In embodiments, the first binding reagent plays a role similar to a “capture reagent” as described in other methods herein, and any characteristic of a capture reagent provided herein may also apply to a first binding reagent.

In embodiments of the method, the first binding reagent binds to a surface marker common to EVs. Surface markers common to EVs are described herein. In embodiments, the marker is a tetraspanin. In embodiments, the tetraspanin is CD9, CD63, or CD81. In embodiments, the first binding reagent binds to a surface marker that is not common to EVs. In embodiments, the first binding reagent binds to a surface marker selected from CD2, CD3, CD4, CD5, CD8, CD10 (NEP), CD11b (ITGAM), CD13 (AAP), CD14, CD15 (SSEA-1), CD16 (FcγRIII), CD18 (ITGB2), CD25 (IL-2Ra), CD26 (DPPIV), CD28, CD29 (ITGB1), CD31 (PECAM-1), CD32b (FcγRII), CD33 (Siglec-3), CD36 (GPIV), CD38, CD40, CD41 (GP2B), CD42b (GP1B), CD42a (GP9), CD44 (HCAM), CD45 (LCA), CD50 (ICAM3), CD54 (ICAM-1), CD61 (GP3A), CD62 (P-Selectin), CD62e (E-Selectin), CD62L (L-Selectin), CD64 (FcγRI), CD66a (CEACAM1), CD66e (CEACAM5), CD68 (LAMP4), CD73 (NT5E), CD95 (FAS), CD105 (Endoglin), CD106 (VCAM-1), CD127 (IL-7Ra), CD141 (Thrombomodulin), CD144 (VE-Cadherin), CD146 (MCAM), CD163, CD166 (ALCAM), CD183 (CXCR3), CD204 (MSR1), CD223 (LAG-3), CD309 (VEGFR2), CD324 (E-Cadherin), CD325 (N-Cadherin), CD326 (EpCAM), CD340 (ERBB2), EphA2, CD202B (TIE2), CX3CR1, ITGB5, HLA-A/B/C, HLA-DR/DP/DQ, ESAM, EGFR, FAPa, FLT-1 (VEGFR1) and GLUT1 (SLC2A1).

In embodiments of the methods herein, each of the first, second and third binding reagents comprises an antibody or antigen binding fragment thereof, antigen, ligand, receptor, oligonucleotide, hapten, epitope, mimitope, lipid binding protein, carbohydrate binding protein, DNA aptamer or RNA aptamer.

The hybridization sequences are complementary sequences between the different oligonucleotide sequences in the constructs. These hybridization sequences may also be referred to as “common overlap” or “overlap” sequences. In embodiments of the method, each hybridization sequence has a length of about 5-10 nucleotides. In embodiments, each hybridization sequence has a length of about 5-7 nucleotides. In embodiments, each hybridization sequence has a length of about 3-15 nucleotides. In embodiments, each hybridization sequence has a length of about 4-13 nucleotides. In embodiments, each hybridization sequence has a length of about 5-12 nucleotides. In embodiments, each hybridization sequence has a length of about 5-11 nucleotides. In embodiments, each hybridization sequence has a length of about 3, 4, 5, 6, 7, 8, 9, 10, 11, 12, 13, 14 or 15 nucleotides. In embodiments, each hybridization sequence is the same length. In embodiments, one or more of the hybridization sequences is a different length. In embodiments, the hybridization includes one or more non-canonical base-pairing.

In embodiments, any of the first, second or third detection sequences can comprise one or more additional barcode sequences. In embodiments, the first detection sequence further comprises a second barcode sequence between the first hybridization sequence and the first amplification primer site. In embodiments, the second barcode sequence of the first detection sequence is a unique molecular identifier. In embodiments, the third detection sequence further comprises a second barcode sequence between the fifth hybridization sequence and the second amplification primer site. In embodiments, the second barcode sequence of the third detection sequence is a unique molecular identifier.

A “barcode sequence” or “barcode oligonucleotide sequence,” as used herein, refers to a short nucleotide (typically between about 5 and about 40 nucleotides in length) that allows a corresponding nucleotide or molecule to be identified. In embodiments, the corresponding nucleotide or molecule is attached to the barcode sequence. In embodiments, the molecule is a peptide, a protein, a protein complex, an antibody, or a vesicle. In embodiments, the barcode sequence is a unique nucleotide identifiable by sequencing. In embodiments, the barcode sequence is hybridizable to a complementary detectable probe. In such embodiments, the complementary detectable probe hybridizes to the barcode sequence, allowing the corresponding nucleotide or molecule to be detected. Barcode technologies are described in, e.g., Winzeler et al., Science 285:901-906 (1999), Eason et al., Proc Natl Acad Sci 101 (30): 11046-11051 (2004), and Fredriksson et al., Nature Methods 4 (4): 327-329 (2007), each of which is herein incorporated by reference in its entirety.

In embodiments of the methods herein, Unique molecular identifiers (UMIs) are used as the barcode. UMIs are a type of molecular barcoding that can provide error correction and increased accuracy during sequencing. These molecular barcodes are short sequences used to uniquely tag each molecule in a sample library. UMIs are used for a wide range of sequencing applications, many around PCR duplicates in DNA and cDNA. UMI deduplication is also useful for RNA-seq gene expression analysis and other quantitative sequencing methods. Typically the length of UMIs is chosen so that the number of possible UMI codes is greater than the number of molecules in the library. In embodiments the length is chosen so that the number of possible UMI codes is much larger than the number of molecules, ensuring that essentially no molecules in the library receive the same UMI code. In embodiments the length is chosen so that the number of possible UMI codes is much larger than the number of sequencing reads to ensure that essentially no sequencing reads have the same UMI codes. In embodiments, the length is chosen so that the number of possible UMI codes is less than the number of sequencing reads to ensure that each molecule is sequenced multiple times. This approach can be used to distinguish rare variants from sequencing errors or synthesis errors. In embodiments, the UMI is used to determine the molecular diversity in a library by counting UMI repeats.

In embodiments the UMI is generated by the inclusion of mixed or degenerate bases during the oligonucleotide synthesis process. In embodiments the UMI is split into 2 or more regions separated by one or more nucleotides that are not part of the UMI. In embodiments, the UMI has a length of about 10-20 nt. In embodiments, the UMI has a length of about 12-18 nt. In embodiments, the UMI has a length of about 15-17 nt.

In embodiments of the method, each barcode has a length of about 5-10 nucleotides. In embodiments, each barcode has a length of about 5-7 nucleotides. In embodiments, each barcode has a length of about 3-15 nucleotides. In embodiments, each barcode has a length of about 4-13 nucleotides. In embodiments, each barcode has a length of about 5-12 nucleotides. In embodiments, each barcode has a length of about 5-11 nucleotides. In embodiments, each barcode has a length of about 3, 4, 5, 6, 7, 8, 9, 10, 11, 12, 13, 14 or 15 nucleotides.

In embodiments, the first detection sequence and third detection sequence have a length of from about 35 to about 55 nucleotides. In embodiments, the first detection sequence and third detection sequence have a length of from about 40 to about 50 nucleotides. In embodiments, the first detection sequence and third detection sequence have a length of from about 30 to about 55 nucleotides. In embodiments, the first detection sequence and third detection sequence have a length of from about 20 to about 60 nucleotides. In embodiments, the first detection sequence and third detection sequence have a length of about 35, 36, 37, 38, 39, 40, 41, 42, 43, 44, 45, 46, 47, 48, 49, 50, 51, 52, 53, 54 or 55 nucleotides. In embodiments, the first detection sequence and third detection sequence are the same length. In embodiments, the first detection sequence and third detection sequence are different lengths.

In embodiments, the second detection sequence has a length of from about 18 to about 38 nucleotides. In embodiments, the second detection sequence has a length of from about 23 to about 33 nucleotides. In embodiments, the second detection sequence has a length of from about 20 to about 32 nucleotides. In embodiments, the second detection sequence has a length of from about 22 to about 30 nucleotides. In embodiments, the second detection sequence has a length of about 18, 19, 20, 21, 22, 23, 24, 25, 26, 27, 28, 29, 30, 31, 32, 33, 34, 35, 36, 37 or 38 nucleotides.

In embodiments, the oligonucleotide insert has a length that allows it to hybridize with the complete sequence of the second detection sequence (splint oligonucleotide) that is not hybridized by the first or third detection sequences. In embodiments, the oligonucleotide insert has a length of about 10 to about 30 nucleotides. In embodiments, the oligonucleotide insert has a length of about 15 to about 25 nucleotides. In embodiments, the oligonucleotide insert has a length of about 15 to about 25 nucleotides.

In embodiments of the method, a single output oligonucleotide attached to the surface of the EV is formed comprising:
(i) hybridizing the first hybridization sequence and the second hybridization sequence, hybridizing the third hybridization sequence and the oligonucleotide insert sequence, and hybridizing the fourth hybridization sequence and the fifth hybridization sequence so that the hybridized first detection sequence is in proximity to one end of the oligonucleotide insert sequence and the fifth hybridization sequence (of the third detection sequence) is in proximity to the other end of the oligonucleotide insert sequence;
(ii) ligating the first hybridization sequence of the hybridized first detection sequence to the hybridized oligonucleotide insert; and
(iii) ligating the hybridized oligonucleotide insert to the fifth hybridization sequence of the hybridized third detection sequence.

In embodiments, the method further comprises amplifying the single output oligonucleotide using a first primer that hybridizes to the first amplification primer site and a second primer that hybridizes to the second amplification primer site. Amplification methods are described herein and are known in the art.

In embodiments of the methods herein, the methods further comprise detecting the amplified single output oligonucleotide. Detection methods are known in the art, and include quantitative PCR (qPCR), array binding and hybridization to labeled probes.

The steps in the above detection method may also be performed to determine the surface markers of an SMDA (such as, for example, and EV), to identify SMDAs that harbor combinations of surface markers, to detect populations of SMDAs having certain surface markers, and/or to detect or quantify multiple populations of SMDAs where each population has a specific set of surface markers. In embodiments of the method, the surface marker displaying agent is an extracellular vesicle (EV). EVs are described herein.

In embodiments, provided herein is a method of determining surface markers of a surface marker displaying agent comprising contacting the surface marker displaying agent with a plurality of unique binding reagents and an oligonucleotide insert.

In embodiments of the methods herein, the methods further comprise amplifying the single output oligonucleotide using a first primer that hybridizes to the first amplification primer site and a second primer that hybridizes to the second amplification primer site. Primers may be synthesized using known methods or purchased from a commercial supplier. Any suitable amplification technique can be used to amplify the output oligonucleotide (or amplicon), including but not limited to, PCR (Polymerase Chain Reaction), LCR (Ligase Chain Reaction), and isothermal amplification methods, e.g., helicase-dependent amplification, rolling circle amplification (RCA), 3SR (Self-Sustained Synthetic Reaction), transcription mediated amplification (TMA), nucleic acid sequence-based amplification (NASBA), signal mediated amplification of RNA technology, strand displacement amplification (SDA), loop-mediated isothermal amplification of DNA (LAMP), isothermal multiple displacement amplification, single primer isothermal amplification, and circular helicase-dependent amplification. In embodiments, the amplification technique is proximity ligation amplification (PLA) using RCA, which is known in the art, and disclosed in International Appl. No. PCT/US2015/030925, published as WO 2015/175856, which is incorporated by reference in its entirety.

In embodiments of the methods herein, the methods further comprise detecting the amplified single output oligonucleotide. Detection methods are known in the art, and include quantitative PCR (qPCR), array binding and hybridization to labeled probes.

In embodiments, each sequencing read contains at least three barcode oligonucleotide sequences, which will be mapped to the identity of the binding reagent. In embodiments, the frequency of a specific combination of binding reagents or entities will be related to the abundance of the three markers (e.g., surface markers on an EV or epitopes on a protein). In embodiments, the abundance of a single marker (e.g., surface markers on an EV or epitopes on a protein) can be determined from the frequency with which the marker is identified from the barcode oligonucleotide sequencing results. In embodiments, multiple binding reagents or entities targeting the same marker can be compared using the barcode oligonucleotide sequencing results. For example, the highest affinity binding reagent or entity can be identified as the binding reagent most represented by its barcode in the sequencing data.

In embodiments, the sequencing is performed with high-throughput sequencing. In embodiments, the sequencing produces at least 106 reads. In embodiments, the sequencing produces at least 107 reads. In embodiments, the sequencing produces at least 108 reads. In embodiments, the sequencing produces at least 109 reads.

### Constructs and Methods for Multimarker Isolation and Analysis of EVs

The present disclosure also provides a method of isolating multimarker extracellular vesicles (EVs), comprising contacting a sample suspected of containing EVs with
a first oligonucleotide-conjugated capture entity,
a second oligonucleotide-conjugated splint entity,
a third oligonucleotide-conjugated staple entity, and
a surface.

In embodiments of the method, the capture entity, splint entity and staple entity are each independently selected from an antibody or antigen binding fragment thereof, antigen, ligand, receptor, oligonucleotide, hapten, epitope, mimitope, lipid binding protein, carbohydrate binding protein, DNA aptamer or RNA aptamer.

In embodiments, the capture entity, splint entity and staple entity are each an antibody or an antibody fragment.

In embodiments of the method, the first oligonucleotide-conjugated capture entity comprises a first oligonucleotide comprising a first Target nucleotide sequence, and a UDG1 labile linkage sequence. In embodiments, the capture entity is conjugated to the first oligonucleotide so that the first Target nucleotide sequence is located between the capture entity and the UDG1 labile linkage sequence.

In embodiments of the method, the second oligonucleotide-conjugated splint entity comprises a second oligonucleotide comprising a second Target nucleotide sequence, a restriction enzyme cleavage site and an additional nucleic acid sequence. In embodiments, the splint entity is conjugated to the second oligonucleotide so that the second Target nucleotide sequence is located between the splint entity and the restriction enzyme cleavage site and the additional nucleic acid sequence is located on the side of the second Target nucleotide sequence (either upstream or downstream) opposite the side that is conjugated to the splint entity.

In embodiments of the method, the third oligonucleotide-conjugated staple entity comprises a third oligonucleotide comprising a third Target nucleotide sequence and a restriction enzyme cleavage site. In embodiments, the staple entity is conjugated to the third oligonucleotide so that the third Target nucleotide sequence is located between the staple entity and the restriction enzyme cleavage site.

In embodiments of the method, complementary DNA sequences on the second oligonucleotide-conjugated splint entity and the third oligonucleotide-conjugated staple entity hybridize to form a double-stranded DNA restriction site. In embodiments, the surface has two capture oligonucleotides immobilized thereon. In embodiments, the first capture oligonucleotide comprises a sequence that is complementary to the additional nucleic acid sequence of the second oligonucleotide of the second oligonucleotide-conjugated splint entity, and is capable of ligating to the end of the third oligonucleotide away from the third oligonucleotide-conjugated staple entity. In embodiments, the second capture oligonucleotide comprises a sequence that is complementary to the UDG1 labile linkage sequence of the first oligonucleotide on the first oligonucleotide-conjugated capture entity.

In embodiments of the method, the capture entity of the first oligonucleotide-conjugated capture entity is conjugated to the 5′ end of the first oligonucleotide. In embodiments, the splint entity of the second oligonucleotide-conjugated splint entity is conjugated to the 3′ end of the second oligonucleotide. In embodiments, the staple entity of the third oligonucleotide-conjugated staple entity is conjugated to the 5′ end of the third oligonucleotide.

In embodiments of the method, each oligonucleotide-conjugated entity is specific for a different EV surface marker. Exemplary EV surface markers are described. In embodiments of the method, at least one oligonucleotide-conjugated entity is specific for an EV surface marker that is associated with a disease or disorder.

In embodiments, the first oligonucleotide of the first oligonucleotide-conjugated capture entity comprises a 5′ first Target nucleotide sequence, and a UDG1 labile linkage sequence located 3′ of the first Target nucleotide sequence.

In embodiments, the third oligonucleotide of the third oligonucleotide-conjugated staple entity is conjugated to the staple entity at the 5′ end of the oligonucleotide and comprises a third Target nucleotide sequence and a restriction enzyme cleavage site positioned 3′ of the third Target nucleotide sequence.

In embodiments, the second oligonucleotide of the second oligonucleotide-conjugated splint entity is conjugated to the antibody at its 3′ end and comprises a second Target nucleotide sequence, a restriction enzyme cleavage site located on the 5′ side of the second Target nucleotide sequence, and an additional nucleic acid sequence positioned 5′ of the restriction enzyme cleavage site.

In embodiments, complementary DNA sequences on the second oligonucleotide-conjugated splint entity and the third oligonucleotide-conjugated staple entity hybridize to form a double-stranded DNA restriction site. In embodiments, the surface has two capture oligonucleotides immobilized thereon. In embodiments, the first capture oligonucleotide comprises a sequence that is complementary to the 5′-most portion of the oligonucleotide of the second oligonucleotide-conjugated splint entity, and is capable of ligating at its 5′ end to the 3′ end of the oligonucleotide of the third oligonucleotide-conjugated staple entity. In embodiments, the capture oligonucleotide comprises a sequence that is complementary at its 3′ end to the UDG1 labile linkage sequence on the first oligonucleotide-conjugated capture entity.

The present disclosure also provides a construct (a set of conjugates) for isolating multimarker extracellular vesicles (EVs) comprising:
a first oligonucleotide-conjugated capture entity,
a second oligonucleotide-conjugated splint entity,
a third oligonucleotide-conjugated staple entity, and
a surface, where each oligonucleotide-conjugated entity is termed a conjugate.

In embodiments of the construct, the first oligonucleotide-conjugated capture entity comprises a first oligonucleotide comprising a first Target nucleotide sequence, and a UDG1 labile linkage sequence. In embodiments, the capture entity is conjugated to the first oligonucleotide so that the first Target nucleotide sequence is located between the capture entity and the UDG1 labile linkage sequence.

In embodiments of the construct, the second oligonucleotide-conjugated splint entity comprises a second oligonucleotide comprising a second Target nucleotide sequence, a restriction enzyme cleavage site and an additional nucleic acid sequence. In embodiments, the splint entity is conjugated to the second oligonucleotide so that the second Target nucleotide sequence is located between the splint entity and the restriction enzyme cleavage site and the additional nucleic acid sequence is located on the side of the second Target nucleotide sequence (either upstream or downstream) opposite the side that is conjugated to the splint entity.

In embodiments of the construct, the third oligonucleotide-conjugated staple entity comprises a third oligonucleotide comprising a third Target nucleotide sequence and a restriction enzyme cleavage site. In embodiments, the staple entity is conjugated to the third oligonucleotide so that the third Target nucleotide sequence is located between the staple entity and the restriction enzyme cleavage site.

In embodiments of the construct, complementary DNA sequences on the second oligonucleotide-conjugated splint entity and the third oligonucleotide-conjugated staple entity hybridize to form a double-stranded DNA restriction site. In embodiments, the surface has two capture oligonucleotides immobilized thereon. In embodiments, the first capture oligonucleotide comprises a sequence that is complementary to the additional nucleic acid sequence of the second oligonucleotide of the second oligonucleotide-conjugated splint entity, and is capable of ligating to the end of the third oligonucleotide away from the third oligonucleotide-conjugated staple entity. In embodiments, the second capture oligonucleotide comprises a sequence that is complementary to the UDG1 labile linkage sequence of the first oligonucleotide on the first oligonucleotide-conjugated capture entity.

In embodiments of the construct, the capture entity of the first oligonucleotide-conjugated capture entity is conjugated to the 5′ end of the first oligonucleotide. In embodiments, the splint entity of the second oligonucleotide-conjugated splint entity is conjugated to the 3′ end of the second oligonucleotide. In embodiments, the staple entity of the third oligonucleotide-conjugated staple entity is conjugated to the 5′ end of the third oligonucleotide.

In embodiments of the construct, each oligonucleotide-conjugated entity is specific for a different EV surface marker. In embodiments, the first oligonucleotide of the first oligonucleotide-conjugated capture entity comprises a 5′ first Target nucleotide sequence, and a UDG1 labile linkage sequence located 3′ of the first Target nucleotide sequence.

In embodiments, the third oligonucleotide of the third oligonucleotide-conjugated staple entity is conjugated to the staple entity at the 5′ end of the oligonucleotide and comprises a third Target nucleotide sequence and a restriction enzyme cleavage site positioned 3′ of the third Target nucleotide sequence.

In embodiments, the second oligonucleotide of the second oligonucleotide-conjugated splint entity is conjugated to the splint entity at the 3′ end of the oligonucleotide and comprises a second Target nucleotide sequence, a restriction enzyme cleavage site located on the 5′ side of the second Target nucleotide sequence, and an additional nucleic acid sequence positioned 5′ of the restriction enzyme cleavage site.

In embodiments, complementary DNA sequences on the second oligonucleotide-conjugated splint entity and the third oligonucleotide-conjugated staple entity hybridize to form a double-stranded DNA restriction site. In embodiments, the surface has two capture oligonucleotides immobilized thereon. In embodiments, the first capture oligonucleotide comprises a sequence that is complementary to the 5′-most portion of the oligonucleotide of the second oligonucleotide-conjugated splint entity, and is capable of ligating at its 5′ end to the 3′ end of the oligonucleotide of the third oligonucleotide-conjugated staple entity. In embodiments, the capture oligonucleotide comprises a sequence that is complementary at its 3′ end to the UDG1 labile linkage sequence on the first oligonucleotide-conjugated capture entity.

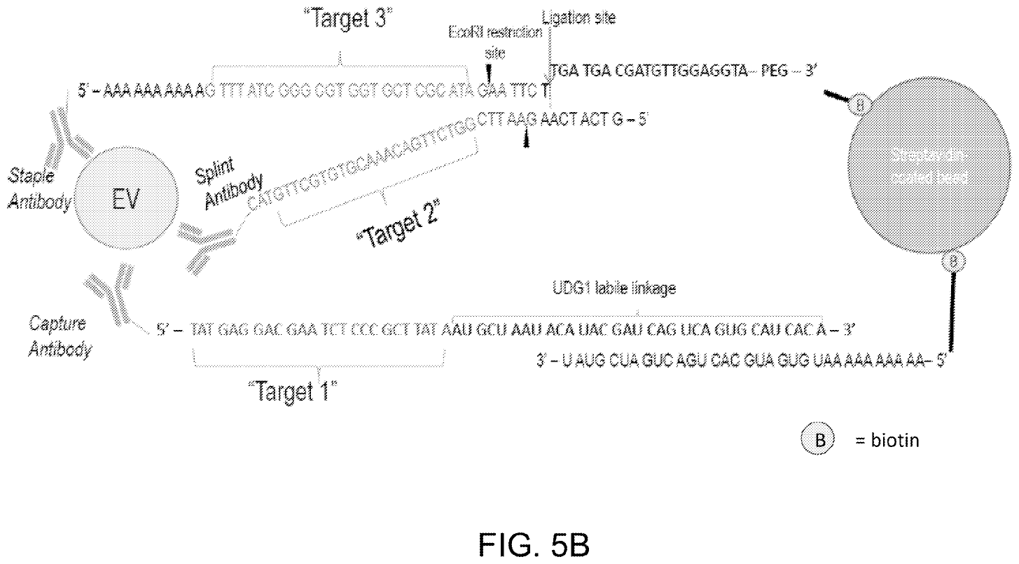

In embodiments, the construct described herein is the construct shown in FIGS. 5A and 5B, where each of the entities is an antibody. In embodiments of the method, the method uses the construct shown in FIG. 5B.

In embodiments of the method or construct, the first Target sequence, second Target sequence and third Target sequence have a length from about 15 to about 25 nucleotides. In embodiments, the first Target sequence, second Target sequence and third Target sequence have a length from about 20 to about 30 nucleotides. In embodiments, the first Target sequence, second Target sequence and third Target sequence have a length from about 17 to about 27 nucleotides. In embodiments, the first Target sequence, second Target sequence and third Target sequence have a length of about 15, 16, 17, 18, 19, 20, 21, 22, 23, 24, 25, 26, 27, 28, 29, 30, 31, 32, 33, 34 or 35 nucleotides.

In embodiments of the method or construct, the restriction enzyme cleavage site is a EcoRI, EcoRII, BamHI, HindIII, TaqI, NotI, HinFI, Sau3AI, PvuII, Smal, HaeIII, HgaI, AluI, EcoRV, EcoP15I, KpnI, PstI, SacI, SalI, ScaI, SpeI, SphI, StuI or XbaI cleavage site.

In embodiments, the UDG1 labile linkage sequence is cleaved by uracil-DNA glycosylase (UDG). UDG removes uracil from DNA. In embodiments, when UDG removes uracil from a segment of DNA, the segment is no longer able to hybridize with its second strand, causing denaturation. In embodiments the UDG1 labile linkage sequence is a DNA sequence comprising uracil nucleotides.

In embodiments of the method or construct, the first oligonucleotide-conjugated capture entity binds to a surface marker common to EVs. Surface markers common to EVs are described herein. In embodiments, the marker is a tetraspanin. In embodiments, the tetraspanin is CD9, CD63, or CD81. In embodiments, the first oligonucleotide-conjugated capture entity binds to a surface marker that is not common to EVs. In embodiments, the first oligonucleotide-conjugated antibody binds to a surface marker selected from CD2, CD3, CD4, CD5, CD8, CD10 (NEP), CD11b (ITGAM), CD13 (AAP), CD14, CD15 (SSEA-1), CD16 (FcγRIII), CD18 (ITGB2), CD25 (IL-2Ra), CD26 (DPPIV), CD28, CD29 (ITGB1), CD31 (PECAM-1), CD32b (FcγRII), CD33 (Siglec-3), CD36 (GPIV), CD38, CD40, CD41 (GP2B), CD42b (GP1B), CD42a (GP9), CD44 (HCAM), CD45 (LCA), CD50 (ICAM3), CD54 (ICAM-1), CD61 (GP3A), CD62 (P-Selectin), CD62e (E-Selectin), CD62L (L-Selectin), CD64 (FcγRI), CD66a (CEACAM1), CD66e (CEACAM5), CD68 (LAMP4), CD73 (NT5E), CD95 (FAS), CD105 (Endoglin), CD106 (VCAM-1), CD127 (IL-7Ra), CD141 (Thrombomodulin), CD144 (VE-Cadherin), CD146 (MCAM), CD163, CD166 (ALCAM), CD183 (CXCR3), CD204 (MSR1), CD223 (LAG-3), CD309 (VEGFR2), CD324 (E-Cadherin), CD325 (N-Cadherin), CD326 (EpCAM), CD340 (ERBB2), EphA2, CD202B (TIE2), CX3CR1, ITGB5, HLA-A/B/C, HLA-DR/DP/DQ, ESAM, EGFR, FAPa, FLT-1 (VEGFR1) and GLUT1 (SLC2A1).

In embodiments of the method of isolating EVs, the Target nucleotide sequences remain intact following EV release and can be hybridized to complementary dye-conjugated in situ probes (complementary to the Target sequence). In embodiments, the in situ probes allow the EV to be fluorescently labeled for multi-color co-localization microscopy/FISH microscopy. In embodiments, a different fluorescent label is attached to each probe directed to a different Target sequence. In embodiments, the probe complementary to the probe complementary to the first Target nucleotide sequence, the probe complementary to the second Target nucleotide sequence and the probe complementary to the third Target nucleotide sequence each have a different fluorescent label. In other embodiments, one or more of the Target sequences can be hybridized to a complementary probe that is biotinylated for subsequent EV pulldown or recapture onto a streptavidin surface for additional analyses including ECL assays.

In embodiments, the multimarker EV isolation method and construct described in this section is used in a method of isolating or detecting an EV requiring a stapling step. Such methods are described below and in and in US Patent Publication 2021/0382043, which is incorporated by reference in its entirety herein. In embodiments, the first oligonucleotide-conjugated capture reagent is used in the isolation of an EV but not used in the detection of an EV.

As used herein, the term “isolating” an EV of interest means to have no more than 5% by weight of any other non-EV components (i.e., unwanted components), and preferably no more than 4%, 3%, 2% or 1% by weight of unwanted components, or preferably no more than 0.8%, 0.6%, 0.4%, 0.2% or 0.1% or less by weight of the unwanted component. The term “isolating” also encompasses amounts of unwanted components that are undetectable by current methods for detecting such components. As used herein, the term “isolating” is synonymous with enriching and purifying.

### Reagents and Entities

The terms capture reagent and binding reagent are generally used herein in the context of methods and kits for detecting EVs, while the terms capture entity, splint entity and staple entity are generally used herein in the context of methods and kits for isolating EVs. In embodiments, these terms are used interchangeably to describe a structure that binds to a target on the surface of an EV. As a specific, non-limiting example, in the methods and kits herein, each of the capture reagent, first binding reagent, second binding reagent, third binding reagent, capture entity, splint entity and staple entity can be an antibody or an antigen binding fragment of an antibody.

In embodiments of the method, the capture reagent, first binding reagent, second binding reagent, capture entity, splint entity and staple entity can be an antibody or antigen binding fragment thereof, antigen, ligand, receptor, oligonucleotide, hapten, epitope, mimitope, lipid binding protein, carbohydrate binding protein, DNA aptamer or RNA aptamer. In embodiments, the entity is a lipid binding protein selected from TIM-4 (T cell immunoglobulin mucin protein 4) and TIM-1 (T cell immunoglobulin mucin protein 1). In embodiments, the entity is a lipid binding protein selected from one of the fatty-acid binding proteins (FABPs): FABP 1, FABP 2, FABP 3, FABP 4, FABP 5, FABP 6, FABP 7, FABP 8, FABP 9, FABP 10, FABP 11, or FABP 12. In embodiments, the entity is a carbohydrate binding protein which is a lectin. In embodiments, the lectin is selected from ConA, LCH, GNA, RCA, PNA, AIL, VVL, WGA, SNA, MAL, MAH, UEA, or AAL. In embodiments, the entity is DNA aptamer or RNA aptamer designed to bind a cell surface marker on the surface of the EV. In embodiments, one or more of the capture entity, splint entity and staple entity are of a different type of entity than the other entities.

### Extracellular Vesicles of Interest

EVs released from a variety of cells target recipient cells for intercellular communication and transfer a subset of genetic materials, proteins, lipids, and metabolites. EVs include a broad spectrum of vesicles secreted by several types of cells and the term is used as a collective one. These include exosomes, ectosomes, oncosomes, shed vesicles, microvesicles, and apoptotic bodies. Thus, EVs represent a broad spectrum of vesicles secreted by several types of cells. Major groups include exosomes (endosomal origin, 40-200 nm), microvesicles/ectosomes (plasma membrane origin, 100-1000 nm) and larger particles such as large-oncosomes (tumor cell origin, >1 μm). The exact definition and nomenclature for each of these general vesicles classes has yet to be fully codified by the field due to their heterogeneous nature, herein, the term “EVs” is as defined by the International Society of Extracellular Vesicles (see Gardiner et al., Journal of Extracellular Vesicles 5 (1): 32945 (2016)).

The isolation and assay methods provided herein enable capture of EVs of interest from the sample, wherein the EVs bear a unique co-localization of surface markers. In embodiments, certain markers can exclude certain unwanted populations of EVs (e.g., use of CD81 as detection marker to exclude platelet derived vesicles). In embodiments, some of the cell-type specific surface markers select EVs of particular origin (i.e., exosomes or ectosomes/microvesicles). In embodiments, the isolation methods exclude very large EVs, apoptotic bodies and cell debris from cell culture supernatants using common techniques like differential centrifugation, ultrafiltration and size-exclusion chromatography but do not otherwise distinguish between small EVs of various origin.

While EVs secreted by neurons and various glial populations have been studied in vitro, isolating populations of EVs from biofluids remains elusive because no method of discriminating these cell-specific EVs has yet been developed. This disclosure provides methods of isolating populations based on the fact that combinations of surface markers define EVs secreted by specific cells such as CNS cells. The methods described herein thus take advantage of the fact that most proteins that are highly expressed on the surface of a particular cell line are also present on the surface of the EVs secreted in cultures of those cells. EVs of interest include cells of the CNS, such as neurons and astrocytes.

In embodiments, the EV of interest is secreted from a cell of the central nervous system (CNS). In embodiments, the cell of the CNS is a neuron, an astrocyte, an oligodendrocyte or a microglia.

In embodiments, the EV comprises a surface marker that is common to EVs. In embodiments, the first marker is common to EVs. In further embodiments, the marker common to EVs is a tetraspanin. Exemplary tetraspanins include CD9, CD37, CD63, CD 81, and CD82.

In embodiments, the EV comprises a surface marker that is a surface adhesion protein. Exemplary surface adhesion proteins include, but are not limited to, EpCAM, E-Cadherin, P-Cadherin, L1CAM, NCAM1, Nectin-4, PECAM and ICAM-1. In embodiments, the EV comprises a surface marker that is a surface receptor. Exemplary surface receptors include, but are not limited to, EGFR, EphA2, TFRC, FasR, and TNFR1. In embodiments, the EV comprises a surface marker that is an endothelial marker. Exemplary endothelial markers include, but are not limited to, PECAM, CD276, TEM7, TEM8, and thrombomodulin. In embodiments, the EV comprises a surface marker that is a tumor antigen. Exemplary tumor antigens include, but are not limited to, CEA, CA19.9, CA50, CA125, CA15.3, mesothelin, cytokeratin-8, E-cadherin, EGFR, EpCAM, EphA2, NCAM, P-cadherin, cMET, Flt-3L, TNFR-2, cKit, ErbB2, and ANXA1. In embodiments, the tumor antigen markers are pancreatic cancer markers. In embodiments, the EV comprises a surface marker that is a platelet EV marker. Exemplary platelet EV markers proteins include, but are not limited to, P-selectin, PECAM, CD63 and CD9.

In embodiments of the disclosure, at least one of EV surface markers is a central nervous system (CNS) cell marker. In additional embodiments, the EV surface marker is specific to a neuron, an astrocyte, an oligodendrocyte or a microglia. In embodiments, the EV surface marker is specific to a neuron. In embodiments, the EV surface marker specific to a neuron is L1CAM, NCAM, NRCAM, CHL1, Glu-R2, neurofascin, DAT1, CD90, CD24 or synaptophysin.

In embodiments, the neuron is a dopaminergic neuron, a GABAergic neuron, a cholinergic neuron, a serotonergic neuron or a glutamatergic neuron.

In embodiments, the EV surface marker is specific to an astrocyte. In embodiments, the surface marker specific to an astrocyte is ALDH1L1, GLT-1, GLAST, CD184, CD44, A2B5, CD80 or CD86. In embodiments, the EV surface marker is specific to an oligodendrocyte. In embodiments, the surface marker specific to an oligodendrocyte is O4, PDGFRa, CSPG4, GD3, MOG, or MBP. In embodiments, the EV surface marker is specific to a microglia. In embodiments, the microglia surface marker is Tmem119, CD11bF4/80, CD68, P2RY12, CXC3R1. In embodiments, the EV surface marker is a disease-specific biomarker.

In embodiments, the EV is an exosome, a micro-vesicle or a large-oncosome.

### Samples

In embodiments of the methods of the disclosure, EVs of interest are isolated from, or detected in, samples using the methods of the disclosure. In embodiments of the disclosure, the sample comprises the EVs of interest and unwanted components. In embodiments of the methods of the disclosure, before contacting the sample with a surface and selectively binding the EV of interest, the sample, e.g., mammalian fluid, secretion, or excretion, is purified by, for instance, differential centrifugation, ultrafiltration, size-exclusion chromatography, affinity, precipitation, or a combination thereof. In embodiments, the unwanted components are soluble in the sample and/or the washing fluid.

In embodiments of the methods of detection described herein, further unwanted components can include, but are not limited to, EVs that do not have the marker that the capture reagent binds to, or EVs that do not have the marker that the binding reagent binds to, or both. In embodiments, the unwanted components include EVs that bind to the capture reagent, but not the binding reagent. In the methods of the disclosure, EVs that bind to the capture reagent, but not the binding reagent, will be eluted following releasing the capture reagent from the surface.

In embodiments of the methods of isolation described herein, further unwanted components can include, but are not limited to, EVs that do not have the marker that the capture entity binds to, or EVs that do not have the marker that the splint entity or staple entity binds to, or EVs that none of the capture entity, splint entity or staple entity binds to. In embodiments, the unwanted components include EVs that bind to the capture entity, but not to one or both of the splint entity or staple entity. In these methods, EVs that bind to the capture entity, but not the splint entity and the staple entity, will be eluted following releasing the capture reagent from the surface.

In embodiments of the methods of the disclosure, the sample comprises EVs produced from a cell differentiated from a cell-line, differentiated from an induced pluripotent stem cell, a primary cell, or a combination thereof. Samples further include cell supernatants, such as those from neuronal and astrocyte cultures, which include at least the following: human cortical neurons differentiated from induced pluripotent stem cells (iPSC) and from the HCN-2 cell line, as well as mature astrocytes differentiated from iPSC and primary human astrocytes. In embodiments, samples include supernatants from oligodendrocytes derived from iPSC cells, which are commercially available, and from cell lines such as HOG or M03.13 which can be differentiated to mature oligodendrocytes using established protocols. Samples further include iPSC derived microglia, which are commercially available, as well as primary microglia which can be expanded in culture.

In embodiments, the sample is a mammalian fluid, secretion, or excretion. In embodiments, the sample is a purified mammalian fluid, secretion, or excretion.

In embodiments, the mammalian fluid, secretion, or excretion is whole blood, plasma, serum, sputum, lachrymal fluid, lymphatic fluid, synovial fluid, pleural effusion, urine, sweat, cerebrospinal fluid, ascites, milk, stool, bronchial lavage, saliva, amniotic fluid, nasal secretions, vaginal secretions, a surface biopsy, sperm, semen/seminal fluid, wound secretions and excretions. In embodiments, the sample is cerebrospinal fluid.

In embodiments, the sample comprises purified EVs. Methods of purification include, but are not limited to, precipitation, ultracentrifugation, size exclusion chromatography, ultrafiltration, or affinity purification. In embodiments, the affinity purification may be performed with magnetic or non-magnetic beads.

Biological samples that may be analyzed include, but are not limited to, physiological samples and/or samples containing suspensions of cells, such as mucosal swabs, tissue aspirates, tissue homogenates, cell cultures, and cell culture supernatant, including cultures of eukaryotic and prokaryotic cells. In embodiments, cells are removed, before contacting the surface with EVs, by, for instance centrifugation or filtration.

### Surfaces and Surface Markers

In embodiments of the disclosure, a sample comprising an EV of interest is contacted with a surface as described herein. The term “contacting” has its ordinary meaning to one of skill in the art. Methods of contacting samples, e.g., liquids, solids, gels, etc., are known to those of ordinary skill in the art.

In embodiments, the EV surface marker to which the capture reagent, capture entity, or first binding reagent binds is common to EVs. Such surface markers include, but are not limited e.g., tetraspanins, such as CD9, CD37, CD63, CD81, CD82. In embodiments, the EV surface marker to which the capture reagent or first binding reagent binds is specific to a central nervous system (CNS) EV. In embodiments, the EV surface marker to which the capture reagent or first binding reagent binds is specific to a neuron EV, an astrocyte EV, an oligodendrocyte EV, or a microglia EV.

In embodiments, the EV surface marker to which the capture reagent, capture entity, or first binding reagent binds is specific to a neuron EV. In embodiments, the neuron-specific EV surface marker to which the capture reagent, capture entity or first binding reagent binds is L1CAM, NCAM, NRCAM, CHL1, Glu-R2, neurofascin, DAT1, CD90, CD24 or synaptophysin.

In embodiments, the neuron that has an EV surface marker to which the capture reagent, capture entity, or first binding reagent binds is a dopaminergic neuron, a GABAergic neuron, a cholinergic neuron, a serotonergic neuron or a glutamatergic neuron.

In embodiments, the EV surface marker to which the capture reagent, capture entity, or first binding reagent binds is specific to an astrocyte EV. In embodiments, the astrocyte-specific EV surface marker to which the capture reagent or first binding reagent binds is ALDH1L1, GLT-1, GLAST, CD184, CD44, A2B5, CD80 or CD86.

In embodiments, the EV surface marker to which the capture reagent, capture entity, or first binding reagent binds is specific to an oligodendrocyte EV. In embodiments, the oligodendrocyte-specific EV surface marker to which the capture reagent or first binding reagent binds is 04, PDGFRa, CSPG4 (NG2, MCSP), GD3, MOG, or MBP.

In embodiments, the EV surface marker to which the capture reagent, capture entity, or first binding reagent binds is specific to a microglia EV. In embodiments, the microglia-specific EV surface marker to which the capture reagent or first binding reagent binds is Tmem119, CD11bF4/80, CD68, P2RY12, or CXC3R1.

In embodiments of the disclosure, the capture reagent, capture entity, or first binding reagent is an antibody to a disease-specific target molecule in or on the surface of the EV. In embodiments, the EV surface marker to which the capture reagent or first binding reagent binds is a cancer antigen. In embodiments, the cancer antigen to which the capture reagent and/or the binding reagent binds is CEA, CA19.9, CA50, CA125, CA15.3, mesothelin, cytokeratin-8, E-cadherin, EGFR, EpCAM, EphA2, NCAM, P-cadherin, cMET, Flt-3L, TNFR-2, cKit, ErbB2, or ANXA1.

In embodiments, the EV surface marker to which the capture reagent, capture entity, or first binding reagent binds is selected from CD2, CD3, CD4, CD5, CD8, CD10 (NEP), CD11b (ITGAM), CD13 (AAP), CD14, CD15 (SSEA-1), CD16 (FcγRIII), CD18 (ITGB2), CD25 (IL-2Ra), CD26 (DPPIV), CD28, CD29 (ITGB1), CD31 (PECAM-1), CD32b (FcγRII), CD33 (Siglec-3), CD36 (GPIV), CD38, CD40, CD41 (GP2B), CD42b (GP1B), CD42a (GP9), CD44 (HCAM), CD45 (LCA), CD50 (ICAM3), CD54 (ICAM-1), CD61 (GP3A), CD62 (P-Selectin), CD62e (E-Selectin), CD62L (L-Selectin), CD64 (FcγRI), CD66a (CEACAM1), CD66e (CEACAM5), CD68 (LAMP4), CD73 (NT5E), CD95 (FAS), CD105 (Endoglin), CD106 (VCAM-1), CD127 (IL-7Ra), CD141 (Thrombomodulin), CD144 (VE-Cadherin), CD146 (MCAM), CD163, CD166 (ALCAM), CD183 (CXCR3), CD204 (MSR1), CD223 (LAG-3), CD309 (VEGFR2), CD324 (E-Cadherin), CD325 (N-Cadherin), CD326 (EpCAM), CD340 (ERBB2), EphA2, CD202B (TIE2), CX3CR1, ITGB5, HLA-A/B/C, HLA-DR/DP/DQ, ESAM, EGFR, FAPa, FLT-1 (VEGFR1) and GLUT1 (SLC2A1).

In embodiments of the methods of the disclosure, the surface comprises an anchoring reagent. In the methods of the disclosure, the anchoring reagent is attached to the surface to allow linker oligonucleotide binding and/or amplicon binding in order to provide an additional indirect attachment point at the surface for the EV of interest. In embodiments, the anchoring reagent includes an oligonucleotide sequence, aptamer, aptamer ligand, antibody, antigen, ligand, receptor, hapten, epitope, or a mimetope; and optionally, the anchoring region can include an aptamer and the anchoring reagent can include an aptamer ligand/target molecule. The anchoring region, in embodiments, comprises a nucleic acid sequence and/or a DNA- or RNA-binding protein. The anchoring reagent comprises, in embodiments, oligonucleotide sequence and the anchoring reagent can include a complementary oligonucleotide sequence. The anchoring reagent, for example, can be a single stranded oligonucleotide sequence or a double stranded oligonucleotide sequence. In embodiments of the disclosure, the anchoring reagent features, etc., are disclosed in International Appl. No. PCT/US2015/030925, published as WO 2015/175856, which is incorporated by reference in its entirety.

In additional embodiments, the amplicon is bound to the anchoring reagent at a position within 10 μm, 5 μm, 1 μm, or 100 nm of the location of the complex comprising the EV of interest on the surface.

Suitable surfaces for use in the methods of the present disclosure are known in the art, including conventional surfaces from the art of binding assays. Suitable surfaces are disclosed, for example, in International Appl. No. PCT/US2015/030925, published as WO 2015/175856. Surfaces may be made from a variety of different materials including polymers (e.g., polystyrene and polypropylene), ceramics, glass, composite materials (e.g., carbon-polymer composites such as carbon-based inks). Suitable surfaces include the surfaces of macroscopic objects such as an interior surface of an assay container (e.g., test tubes, cuvettes, flow cells, FACS cell sorter, cartridges, wells in a multi-well plate, etc.), slides, assay chips (such as those used in gene or protein chip measurements), pins or probes, beads, filtration media, lateral flow media (for example, filtration membranes used in lateral flow test strips), etc.

Suitable surfaces also include particles (including but not limited to colloids or beads) commonly used in other types of particle-based assays e.g., magnetic, polypropylene, and latex particles, hydrogels, e.g. agarose, materials typically used in solid-phase synthesis e.g., polystyrene and polyacrylamide particles, and materials typically used in chromatographic applications e.g., silica, alumina, polyacrylamide, polystyrene. The materials may also be a fiber such as a carbon fibril. Microparticles may be inanimate or alternatively, may include animate biological entities such as cells, viruses, bacterium and the like. A particle used in the present method may be comprised of any material suitable for attachment to one or more capture or anchoring reagents, and that may be collected via, e.g., centrifugation, gravity, filtration or magnetic collection. A wide variety of different types of particles that may be attached to capture or anchoring reagents are sold commercially for use in binding assays. These include non-magnetic particles as well as particles comprising magnetizable materials which allow the particles to be collected with a magnetic field. In one embodiment, the particles are comprised of a conductive and/or semiconductive material, e.g., colloidal gold particles. The microparticles may have a wide variety of sizes and shapes. By way of example and not limitation, microparticles may be between 5 nanometers and 100 micrometers. Preferably microparticles have sizes between 20 nm and 10 micrometers. The particles may be spherical, oblong, rod-like, etc., or they may be irregular in shape.

The particles used in the present method may be coded to allow for the identification of specific particles or subpopulations of particles in a mixture of particles. The use of such coded particles has been used to enable multiplexing of assays employing particles as solid phase supports for binding assays. In one approach, particles are manufactured to include one or more fluorescent dyes and specific populations of particles are identified based on the intensity and/or relative intensity of fluorescence emissions at one or more wave lengths. This approach has been used in the Luminex xMAP systems (see, e.g., U.S. Pat. No. 6,939,720) and the Becton Dickinson Cytometric Bead Array systems. Alternatively, particles may be coded through differences in other physical properties such as size, shape, imbedded optical patterns and the like. One or more particles provided in a mixture or set of particles may be coded to be distinguishable from other particles in the mixture by virtue of particle optical properties, size, shape, imbedded optical patterns and the like.

In a specific embodiment, the methods of the disclosure can be used in a multiplexed format by binding a plurality of different analytes to a plurality of capture reagents for those analytes, the capture reagent being immobilized on coded beads, such that the coding of the beads identifies the capture reagent (and analyte target) for a specific bead. The method may further comprise counting the number of beads that have a bound analyte (using the detection approaches described herein).

Alternatively or additionally, the capture reagents can be bound, directly or indirectly, to different discrete binding domains on one or more solid phases, e.g., as in a binding array wherein the binding domains are individual array elements, or in a set of beads wherein the binding domains are the individual beads, such that discrete assay signals are generated on and measured from each binding domain. If capture reagents for different analytes are immobilized in different binding domains, the different analytes bound to those domains can be measured independently. In one example of such an embodiment, the binding domains are prepared by immobilizing, on one or more surfaces, discrete domains of capture reagents that bind analytes of interest. Optionally, the surface(s) may define, in part, one or more boundaries of a container (e.g., a flow cell, well, cuvette, etc.) which holds the sample or through which the sample is passed. In a preferred embodiment, individual binding domains are formed on electrodes for use in electrochemical or electrochemiluminescence assays. Multiplexed measurement of analytes on a surface comprising a plurality of binding domains using electrochemiluminescence has been used in the Meso Scale Diagnostics, LLC., MULTI-ARRAY® and SECTOR® Imager line of products (see, e.g., U.S. Pat. Nos. 7,842,246 and 6,977,722, the disclosures of which are incorporated herein by reference in their entireties).

Still further, the capture reagents can be bound, directly or indirectly, to an electrode surface, which optionally includes different discrete binding domains, as described above. The electrode surface can be a component of a multi-well plate and/or a flow cell. Electrodes can comprise a conductive material, e.g., a metal such as gold, silver, platinum, nickel, steel, iridium, copper, aluminum, a conductive allow, or the like. They may also include oxide coated metals, e.g., aluminum oxide coated aluminum. The electrode can include working and counter electrodes which can be made of the same or different materials, e.g., a metal counter electrode and carbon working electrode. In one specific embodiment, electrodes comprise carbon-based materials such as carbon, carbon black, graphitic carbon, carbon nanotubes, carbon fibrils, graphite, graphene, carbon fibers and mixtures thereof. In one embodiment, the electrodes comprise elemental carbon, e.g., graphitic, carbon black, carbon nanotubes, etc. Advantageously, they may include conducting carbon-polymer composites, conducting particles dispersed in a matrix (e.g., carbon inks, carbon pastes, metal inks, graphene inks), and/or conducting polymers. One specific embodiment of the disclosure is an assay module, preferably a multi-well plate, having electrodes (e.g., working and/or counter electrodes) that comprise carbon, e.g., carbon layers, and/or screen-printed layers of carbon inks.

In embodiments, the capture reagent is attached to the surface via a pair of short complementary oligonucleotides (one attached to the surface, the other attached to the capture reagent) that form stable duplexes in common biological buffers but can be denatured in a low salt buffer, and modestly elevated temperature is used to allow the capture reagent, e.g., antibody to be released. In embodiments, a restriction site in the complementary oligonucleotides that is cleaved by a restriction endonuclease is used. This has the advantage of being completely orthogonal to the denaturation that will be used, in embodiments, to purposely elute the stapled EVs, though a second restriction enzyme can also be used to elute the stapled EVs. Stapling sequence, diluents, and procedure are optimized to maximize retention of stapled EVs and minimize retention of non-stapled EVs. In embodiments, the captured EVs are co-labeled with STAG-labeled detection antibodies, and the electrochemiluminescence (ECL) signal is compared with the ECL signal generated with and without elution, or with specific stapling and irrelevant stapling.

In embodiments of the disclosure, the capture reagent binds to a first surface marker on the EV and the binding reagent binds to a second surface marker on the EV.

### Assaying the EV

In embodiments of the disclosure, the EV of interest is assayed. In embodiments, the assay is an ultrasensitive assay. In embodiments of the disclosure, the EV of interest is assayed while bound to the surface, either by both attachment points, e.g., by the capture reagent and by the binding reagent/anchoring reagent, or after the capture reagent is released from the surface. In embodiments, the assaying comprises contacting a detectably labeled oligonucleotide with the surface, wherein the oligonucleotide is complementary to the amplicon. In embodiments, the binding reagent is detectably labeled.

In embodiments of the disclosure, bound EVs of interest are subjected to a measuring step, which are known to those of skill in the art, for example, as disclosed in International Appl. No. PCT/US2015/030925, published as WO 2015/175856, which is incorporated by reference in its entirety. In embodiments, EVs of interest are measured using an ultrasensitive assay format for soluble proteins that marries a variation of proximity ligation amplification (PLA) with ECL detection to provide state-of-the-art sensitivity. The measuring step of the method can comprise imaging an optical signal from the surface to generate an image that consists of a plurality of pixels, wherein each resolvable binding region maps to one or more pixels or groups of pixels in the image. Image analysis to identify pixels or sets of pixels having a signal indicative of a binding event (detection complex) can be accomplished using art recognized methods.

In one embodiment, the resolvable binding regions are elements of an array. In embodiments, the array is an array of micro-wells or nanowells, e.g., individual depressions or wells of a unitary substrate. Preferably, the volume of the wells is less than 500 μL, 300 μL, 150 μL, 100 μL, 10 μL, 1 μL, 100 nL, preferably less than 50 nL. In one embodiment, the volume of the wells ranges from approximately 10 aL-100 pL. Optionally, the wells may be configured to hold a microparticle.

In one embodiment, at least 50% of the resolvable binding regions positioned on a substrate and addressed during an assay contain either zero or one analyte molecule. Preferably, at least 80%, more preferably at least 95%, and most preferably at least 99% of the resolvable binding regions contain either zero or one analyte molecule. The concentration of analyte molecules in the sample is determined at least in part using a calibration curve, a Poisson distribution analysis and/or a Gaussian distribution analysis of the number of binding regions that contain at least one or one analyte molecule. In a specific embodiment, the surface comprises a plurality of particles each including a plurality of capture reagents for an analyte molecule and the plurality of particles is distributed across a plurality of resolvable binding regions (e.g., an array of micro- or nano-wells). Therefore, the method includes: (i) binding one or more analyte molecules to one or more capture reagents on the surface, (ii) distributing the plurality of particles across an array of resolvable binding regions; and (iii) determining the presence or absence of an analyte molecule in each resolvable binding regions, so as to identify the number of binding domains that contain an analyte molecule and/or the number of binding domains that do not contain an analyte molecule.

Alternatively, labels used to detect analyte molecules can be fluorescent species that can be used in single molecule fluorescence detection, e.g., fluorescence correlation spectroscopy, and/or fluorescence cross-correlation spectroscopy. Single molecule fluorescence detection comprises flowing an eluent that includes a detectable species through a capillary, focusing a light source on a volume within the capillary to create an interrogation zone and observing the interrogation zone with a light detector to detect the passage of fluorescent molecules through the interrogation zone.

In one embodiment, the EV of interest in the sample may be measured using electrochemiluminescence-based assay formats, e.g., electrochemiluminescence (ECL) based immunoassays. Species that can be induced to emit ECL (ECL-active species) have been used as ECL labels, e.g., i) organometallic compounds where the metal is from, for example, the noble metals of group VIII, including Ru-containing and Os-containing organometallic compounds such as the tris-bipyridyl-ruthenium (RuBpy) moiety and ii) luminol and related compounds. Species that participate with the ECL label in the ECL process are referred to herein as ECL coreactants. Commonly used coreactants include tertiary amines (e.g., see U.S. Pat. No. 5,846,485), oxalate, and persulfate for ECL from RuBpy and hydrogen peroxide for ECL from luminol (see, e.g., U.S. Pat. No. 5,240,863). The light generated by ECL labels can be used as a reporter signal in diagnostic procedures (Bard et al., U.S. Pat. No. 5,238,808, herein incorporated by reference). For instance, an ECL label can be covalently coupled to a binding agent such as an antibody, nucleic acid probe, receptor or ligand; the participation of the binding reagent in a binding interaction can be monitored by measuring ECL emitted from the ECL label. Alternatively, the ECL signal from an ECL-active compound may be indicative of the chemical environment (see, e.g., U.S. Pat. No. 5,641,623 which describes ECL assays that monitor the formation or destruction of ECL coreactants).

The methods of the disclosure may be applied to singleplex or multiplex formats where multiple assay measurements are performed on a single sample. Multiplex measurements that can be used with the disclosure include, but are not limited to, multiplex measurements i) that involve the use of multiple sensors; ii) that use discrete assay domains on a surface (e.g., an array) that are distinguishable based on location on the surface; iii) that involve the use of reagents coated on particles that are distinguishable based on a particle property such as size, shape, color, etc.; iv) that produce assay signals that are distinguishable based on optical properties (e.g., absorbance or emission spectrum) or v) that are based on temporal properties of assay signal (e.g., time, frequency or phase of a signal).

The disclosure includes methods for detecting and counting individual detection complexes. In a specific embodiment, the surface can comprise a plurality of capture reagents for one or more EVs that are present in a sample and the plurality of capture reagents are distributed across a plurality of resolvable binding regions positioned on the surface. Under the conditions used to carry out and analyze a measurement, a “resolvable binding region” is the minimal surface area associated with an individual binding event that can be resolved and differentiated from another area in which an additional individual binding event is occurring. Therefore, the method consists of binding one or more EVs of interest to one or more capture reagents on the surface, determining the presence or absence of the EV in a plurality of resolvable binding regions on the surface, and identifying the number of resolvable binding regions that contain an EV of interest and/or the number of analyte domains that do not contain an EV of interest.

The resolvable binding regions can be optically interrogated, in whole or in part, i.e., each individual resolvable binding region can be individually optically interrogated and/or the entire surface comprising a plurality of resolvable binding regions can be imaged and one or more pixels or groupings of pixels within that image can be mapped to an individual resolvable binding region. A resolvable binding region may also be a microparticle within a plurality of microparticles. The resolvable binding regions exhibiting changes in their optical signature can be identified by a conventional optical detection system. Depending on the detected species (e.g., type of fluorescence entity, etc.) and the operative wavelengths, optical filters designed for a particular wavelength can be employed for optical interrogation of the resolvable binding regions. In embodiments where optical interrogation is used, the system can comprise more than one light source and/or a plurality of filters to adjust the wavelength and/or intensity of the light source. In some embodiments, the optical signal from a plurality of resolvable binding regions is captured using a CCD camera. Other non-limiting examples of camera imaging systems that can be used to capture images include charge injection devices (CIDs), complementary metal oxide semiconductors (CMOSs) devices, scientific CMOS (sCMOS) devices, and time delay integration (TDI) devices, as will be known to those of ordinary skill in the art. In some embodiments, a scanning mirror system coupled with a photodiode or photomultiplier tube (PMT) can be used for imaging.

Additional methods of interrogating whole EVs are known in the art, such as by bioluminescence, and nuclear magnetic resonance (NMR). In additional embodiments, for example, the EV of interest is assessed by quantitative polymerase chain reaction, next generation sequencing, or both.

### Controls

In an additional embodiment, the assay formats described herein further include one or more control assays. A negative control can be included on a binding domain which includes a capture reagent or capture entity that does not have a corresponding detection antibody, thereby providing a consistent background signal for all samples. Measurement of signal above a preset threshold value can indicate improper assay processing or the presence of a sample-dependent matrix effect causing non-specific binding of labeled detection probe. Moreover, a specimen control can also be included in the assay for a human target antigen (such as a secreted or intracellular protein) that performs multiple control functions. A positive signal will indicate the presence of human material, and therefore test for sample addition and quality. Measurement of a signal below a predefined threshold would indicate that no sample was added, that a failure in the reagents or process occurred, or that substances that interfere with amplification or detection are present. In addition to internal controls, external positive and negative controls can also be used with the method and/or kit. The negative control comprises a representative matrix without any target proteins.

In embodiments, a control EV is used to establish the performance of the assay or provide a reliable sample for normalizing data, or both. In embodiments, control EVs facilitate comparison of results between plates or experiments, or both. In embodiments, a control EV is used for correction of nonlinearity of an assay at upper and lower ends of the calibration curve. For example, it may be advantageous to utilize a synthetic EV, which allows for selection of surface antigens, and the copy number can be tuned to match the biological material of interest. In embodiments, the control EV has similar size and density to the EV of interest. In embodiments, the synthetic EV is produced using polymer beads of similar size and density to small EVs. In embodiments, tetraspanin proteins are attached to the surface of the synthetic EV.

In embodiments, well-characterized, biologically-derived EVs are used as controls. In embodiments, control EVs are produced from a cell line selected for its efficiency at producing EVs. In embodiments, EVs from cell lines or biofluids are used as negative controls, such as from platelets, PBMCs, THP-1 cells, Expi293 cells, and HCT-15 cells. In embodiments, synthetic EVs, such as unilamellar vesicles or beads that have similar physiochemical properties as the EVs of interest, are used as controls.

### EV Surface Markers for Neuro-COVID

SARS-COV-2, which causes COVID-19, has been shown to cause neurological complications in a significant portion of COVID-19 patients. Patients having these neurological symptoms are sometimes referred to as neuro-COVID patients. In embodiments, the methods, constructs and kits described herein can be used to determine if a subject has neuro-COVID, previously had neuro-COVID or is at risk of developing neuro-COVID complications.

In embodiments the subject is diagnosed with neuro-COVID if elevated levels of extracellular vesicles (EVs) having certain neurological surface markers are found in a sample taken from the subject. A sample taken from a subject may include, for example, cerebral spinal fluid (CSF), plasma, serum, blood, brain tissue, saliva, or urine. In embodiment, the sample is a CSF or plasma sample. In embodiments, the subject is diagnosed with neuro-COVID if elevated levels of EVs having microglial surface markers are found in a sample taken from the subject. In embodiments, the subject is diagnosed with neuro-COVID if elevated levels of EVs having microglial surface markers, but not elevated levels of neuronal EVs or astrocyte EVs, are found in a sample taken from the subject.

In embodiments, the subject is diagnosed with neuro-COVID if elevated levels of EVs having microglial surface markers selected from one or more of CX3CR1, MHC Class II, CD33, CD36, ITGB5, IBA1, CD14, CD40, CD64, Stabilin-1 or GPNMB are found in a sample taken from the subject. In embodiments, the subject is diagnosed with neuro-COVID if elevated levels of EVs having microglial surface markers selected from one or more of ITGB5, CX3CR1, CD40, CD64, HLA-DR/DP/DQ or IBA1 are found in a sample taken from the subject. In embodiments, the subject is diagnosed with neuro-COVID if elevated levels of EVs having the microglial surface marker IBA1 are found in a sample taken from the subject. Samples that can be assayed as part of this embodiment are described elsewhere herein.

### III. Kits

The disclosure further provides a kit for detecting an EV in a sample comprising, in one or more vials, containers, or compartments:
(i) a capture reagent;
(ii) a first binding reagent that binds a first surface marker of the EV, wherein the first binding reagent comprises a first detection sequence that comprises a first hybridization sequence, and a first amplification primer site;
(iii) a second binding reagent that binds a second surface marker of the EV, wherein the second binding reagent comprises a second detection sequence that comprises a second hybridization sequence, a third hybridization sequence, and a fourth hybridization sequence;
(iv) a third binding reagent comprising a third detection sequence, wherein the third detection sequence comprises a fifth hybridization sequence, and a second amplification primer site; and
(v) an oligonucleotide insert comprising an oligonucleotide insert sequence.
In embodiments, the third detection sequence is releasably bound to the surface. In embodiments, the kit may further comprise a capture reagent releasably bound to the surface.

In embodiments of the kit, the first hybridization sequence and the second hybridization sequence are complementary. In embodiments, the fourth hybridization sequence and the fifth hybridization sequence are complementary. In embodiments, the third hybridization sequence is complementary to the oligonucleotide insert sequence.

In embodiments of the kit, the capture reagent is bound to a surface. In embodiments, the third binding reagent is bound to the same surface as the capture reagent. In embodiments, the third binding reagent binds a third surface marker of the EV.

In embodiments, the kit further comprises a first primer complementary to the first amplification primer site and a second primer complementary to the second amplification primer site. In embodiments, the kit comprises multiple different capture reagents and multiple different first, second, and third binding reagents to allow combinatorial analysis of markers.

In embodiments of the kit, the capture reagent binds to a surface marker common to EVs. In embodiments, the marker is a tetraspanin. In embodiments, the tetraspanin is CD9, CD63, or CD81. In embodiments, the capture reagent binds to a surface marker that is not common to EVs. In embodiments, the capture reagent binds to a surface marker selected from CD2, CD3, CD4, CD5, CD8, CD10 (NEP), CD11b (ITGAM), CD13 (AAP), CD14, CD15 (SSEA-1), CD16 (FcγRIII), CD18 (ITGB2), CD25 (IL-2Ra), CD26 (DPPIV), CD28, CD29 (ITGB1), CD31 (PECAM-1), CD32b (FcγRII), CD33 (Siglec-3), CD36 (GPIV), CD38, CD40, CD41 (GP2B), CD42b (GP1B), CD42a (GP9), CD44 (HCAM), CD45 (LCA), CD50 (ICAM3), CD54 (ICAM-1), CD61 (GP3A), CD62 (P-Selectin), CD62e (E-Selectin), CD62L (L-Selectin), CD64 (FcγRI), CD66a (CEACAM1), CD66e (CEACAM5), CD68 (LAMP4), CD73 (NT5E), CD95 (FAS), CD105 (Endoglin), CD106 (VCAM-1), CD127 (IL-7Ra), CD141 (Thrombomodulin), CD144 (VE-Cadherin), CD146 (MCAM), CD163, CD166 (ALCAM), CD183 (CXCR3), CD204 (MSR1), CD223 (LAG-3), CD309 (VEGFR2), CD324 (E-Cadherin), CD325 (N-Cadherin), CD326 (EpCAM), CD340 (ERBB2), EphA2, CD202B (TIE2), CX3CR1, ITGB5, HLA-A/B/C, HLA-DR/DP/DQ, ESAM, EGFR, FAPa, FLT-1 (VEGFR1) and GLUT1 (SLC2A1).

The disclosure further provides a kit for detecting an EV in a sample comprising, in one or more vials, containers, or compartments at least three unique binding reagents and an oligonucleotide insert.

In embodiments of the kit, each unique binding reagent comprises a detection sequence comprising a unique barcode oligonucleotide sequence. In embodiments, when at least three unique binding reagents bind to three unique surface markers of the surface marker displaying agent, an output oligonucleotide is generated that comprises the barcode oligonucleotide sequences of each of the three unique binding reagents. In embodiments, the output oligonucleotide is capable of being sequenced to identify the three unique surface markers of the surface marker displaying agent.

In embodiments, the plurality of binding reagents comprises:
a. a first binding reagent comprising a first detection sequence that comprises a first hybridization sequence, and a first amplification primer site;
b. a second binding reagent comprising a second detection sequence that comprises a second hybridization sequence, a third hybridization sequence, and a fourth hybridization sequence; and
c. a third binding reagent comprising a third detection sequence that comprises a fifth hybridization sequence, and a second amplification primer site,
wherein the first hybridization sequence and the second hybridization sequence are complementary.

In embodiments, the fourth hybridization sequence and the fifth hybridization sequence are complementary. In embodiments, the third hybridization sequence is complementary to the oligonucleotide insert sequence.

In embodiments of the kit, the first binding reagent binds to a surface marker common to EVs. In embodiments, the marker is a tetraspanin. In embodiments, the tetraspanin is CD9, CD63, or CD81. In embodiments, the first binding reagent binds to a surface marker that is not common to EVs. In embodiments, the first binding reagent binds to a surface marker selected from CD2, CD3, CD4, CD5, CD8, CD10 (NEP), CD11b (ITGAM), CD13 (AAP), CD14, CD15 (SSEA-1), CD16 (FcγRIII), CD18 (ITGB2), CD25 (IL-2Ra), CD26 (DPPIV), CD28, CD29 (ITGB1), CD31 (PECAM-1), CD32b (FcγRII), CD33 (Siglec-3), CD36 (GPIV), CD38, CD40, CD41 (GP2B), CD42b (GP1B), CD42a (GP9), CD44 (HCAM), CD45 (LCA), CD50 (ICAM3), CD54 (ICAM-1), CD61 (GP3A), CD62 (P-Selectin), CD62e (E-Selectin), CD62L (L-Selectin), CD64 (FcγRI), CD66a (CEACAM1), CD66e (CEACAM5), CD68 (LAMP4), CD73 (NT5E), CD95 (FAS), CD105 (Endoglin), CD106 (VCAM-1), CD127 (IL-7Ra), CD141 (Thrombomodulin), CD144 (VE-Cadherin), CD146 (MCAM), CD163, CD166 (ALCAM), CD183 (CXCR3), CD204 (MSR1), CD223 (LAG-3), CD309 (VEGFR2), CD324 (E-Cadherin), CD325 (N-Cadherin), CD326 (EpCAM), CD340 (ERBB2), EphA2, CD202B (TIE2), CX3CR1, ITGB5, HLA-A/B/C, HLA-DR/DP/DQ, ESAM, EGFR, FAPa, FLT-1 (VEGFR1) and GLUT1 (SLC2A1).

In embodiments of any of the above kits, the kit further comprises a ligase.

The disclosure further provides a kit for isolating multimarker extracellular vesicles (EVs) comprising, in one or more vials, containers, or compartments:
a first oligonucleotide-conjugated capture entity,
a second oligonucleotide-conjugated splint entity,
a third oligonucleotide-conjugated staple entity, and
a surface.

In embodiments of the kit, the capture entity, splint entity and staple entity are each independently selected from an antibody or antigen binding fragment thereof, antigen, ligand, receptor, oligonucleotide, hapten, epitope, mimitope, lipid binding protein, carbohydrate binding protein, DNA aptamer or RNA aptamer. In embodiments, one or more of the capture entity, splint entity and staple entity are of a different type of entity than the other entities.

In embodiments of the kit, the capture entity, splint entity and staple entity are each an antibody or an antibody fragment.

In embodiments of the kit, the first oligonucleotide-conjugated capture entity comprises a first oligonucleotide comprising a first Target nucleotide sequence, and a UDG1 labile linkage sequence. In embodiments, the capture entity is conjugated to the first oligonucleotide so that the first Target nucleotide sequence is located between the capture entity and the UDG1 labile linkage sequence.

In embodiments of the kit, the second oligonucleotide-conjugated splint entity comprises a second oligonucleotide comprising a second Target nucleotide sequence, a restriction enzyme cleavage site and an additional nucleic acid sequence. In embodiments, the splint entity is conjugated to the second oligonucleotide so that the second Target nucleotide sequence is located between the splint entity and the restriction enzyme cleavage site, and the additional nucleic acid sequence is located on the side of the second Target sequence (either upstream or downstream) opposite the side that is conjugated to the splint entity.

In embodiments of the kit, the third oligonucleotide-conjugated staple entity comprises a third oligonucleotide comprising a third Target nucleotide sequence and a restriction enzyme cleavage site. In embodiments, the staple entity is conjugated to the third oligonucleotide so that the third Target nucleotide sequence is located between the staple entity and the restriction enzyme cleavage site.

In embodiments of the kit, complementary DNA sequences on the second oligonucleotide-conjugated splint entity and the third oligonucleotide-conjugated staple entity hybridize to form a double-stranded DNA restriction site. In embodiments, the surface has two capture oligonucleotides immobilized thereon. In embodiments, the first capture oligonucleotide comprises a sequence that is complementary to the additional nucleic acid sequence of the second oligonucleotide of the second oligonucleotide-conjugated splint entity, and is capable of ligating to the end of the third oligonucleotide away from the third oligonucleotide-conjugated staple entity. In embodiments, the second capture oligonucleotide comprises a sequence that is complementary to the UDG1 labile linkage sequence of the first oligonucleotide on the first oligonucleotide-conjugated capture entity.

In embodiments of the kit, the capture entity of the first oligonucleotide-conjugated capture entity is conjugated to the 5′ end of the first oligonucleotide. In embodiments, the splint entity of the second oligonucleotide-conjugated splint entity is conjugated to the 3′ end of the second oligonucleotide. In embodiments, the staple entity of the third oligonucleotide-conjugated staple entity is conjugated to the 5′ end of the third oligonucleotide.

In embodiments, the third oligonucleotide of the third oligonucleotide-conjugated staple entity is conjugated to the staple entity at the 5′ end of the oligonucleotide and comprises a third Target nucleotide sequence and a restriction enzyme cleavage site positioned 3′ of the third Target nucleotide sequence.

In embodiments, the second oligonucleotide of the second oligonucleotide-conjugated splint entity is conjugated to the splint entity at the 3′ end of the oligonucleotide and comprises a second Target nucleotide sequence, a restriction enzyme cleavage site located on the 5′ side of the second Target nucleotide sequence, and an additional nucleic acid sequence positioned 5′ of the restriction enzyme cleavage site. In embodiments, complementary DNA sequences on the second oligonucleotide-conjugated splint entity and the third oligonucleotide-conjugated staple entity hybridize to form a double-stranded DNA restriction site.

In embodiments, the surface has two capture oligonucleotides immobilized thereon. In embodiments, the first capture oligonucleotide comprises a sequence that is complementary to the 5′-most portion of the oligonucleotide of the second oligonucleotide-conjugated splint entity, and is capable of ligating at its 5′ end to the 3′ end of the oligonucleotide of the third oligonucleotide-conjugated staple entity. In embodiments, the second capture oligonucleotide comprises a sequence that is complementary at its 3′ end to the UDG1 labile linkage sequence on the first oligonucleotide-conjugated capture entity.

In embodiments of the kit, each oligonucleotide-conjugated entity is specific for a different EV surface marker. In embodiments, the first oligonucleotide of the first oligonucleotide-conjugated capture entity comprises a 5′ first Target nucleotide sequence, and a UDG1 labile linkage sequence located 3′ of the first Target nucleotide sequence.

In embodiments of the kits for isolating multimarker EVs, the kits further comprise dye-conjugated in situ probes that are complementary to the Target sequences. In embodiments, a different fluorescent label is attached to each probe directed to a different Target sequence. In other embodiments, the kits further comprise probes that are biotinylated and are complementary to the Target sequences. These biotinylated probes allow for subsequent EV pulldown or recapture onto a streptavidin surface for additional analyses including ECL assays.

In embodiments of any of the above kits, the surface comprises a particle, a bead, or a surface of a culture dish, culture well, or plate. Suitable surfaces are described herein.

In embodiments of any of the above kits, the kit comprises one or more buffers. In embodiments, the kit comprises one or more of a wash buffer, an assay buffer, and a read buffer. In embodiments, the same buffer can be used for the wash, assay, and detection (i.e., “read”) steps. In embodiments, the kit comprises a Tris buffer and/or a phosphate buffer. Non-limiting examples of wash buffers, assay buffers, and/or read buffers include phosphate buffer, Tris buffer, HEPES buffer, and the like. In embodiments, the wash buffer and/or the read buffer comprises a surfactant. In some embodiments, the surfactant is TRITON-X. In embodiments, the surfactant is TWEEN-20. In embodiments, the wash buffer and/or the read buffer comprises a co-reactant. In embodiments, the co-reactant is tripropylamine (TPA). In embodiments, the read buffer is a Tris buffer comprising TRITON-X and TPA.

### Automated High Throughput EV Detection

The disclosure further provides an automated version of the detection methods of the disclosure using a high-throughput robotic liquid handling system. This system allows simultaneous preparation of up to 480 samples with accuracy and reproducibility unmatched by a human operator. In embodiments, the automated system is a free-standing, fully integrated system for carrying out immunoassays using ECL technology. This system, capable of simultaneously running up to five 96-well assay plates, consists of a robotic lab automation workstation for liquid handling and plate manipulation, physically integrated with an ECL reader.

In embodiments, the workflow conducts the methods of the disclosure with minimal human intervention. In embodiments, a single 96-well source plate is loaded with plasma samples in each well. Sample is dispensed along with appropriate diluents into the desired capture plate having capture reagents attached to the surface. Plates are incubated on shakers while EVs are captured. Next, the plates are washed and the binding reagents are then added and incubated. Plates are washed again followed by addition of oligonucleotide insert and ligase. Plates are washed again followed by addition amplification primers and reagents for generating a single output oligonucleotide. The operator only has to set up the source plate, and load reagents into the instrument. No further intervention is required. In embodiments, the procedure is run three days in a row on the same samples with the same plate and reagent lots to assess run-to-run variability.

### Screening for Multiple Markers to a Same Target

One of the challenges associated with multi-marker isolation (i.e., using two or more capture and/or detection reagents) of target molecules, for example, macromolecules such as proteins, protein complexes, or extracellular vesicles (EVs), is the need to efficiently identify multi-marker signatures that correspond to the targets of interest. Multiple markers may need to be identified in any immunoassay requiring three or more binding reagents on a single protein, protein complex, or large macromolecule such as EV. For example, multiple binding reagents or entities may be desired for binding to different epitopes on the same protein. Multiple binding reagents or entities may also be desired for binding to different surface markers on the same EV. In another example, multiple binding reagents or entities may be desired for binding to the same epitope in a multimeric target (e.g., protein or protein complex), and it may be desirable to use one or more of the same binding reagents or entities for binding to the same epitope in different monomers of the multimeric target. The present disclosure provides methods of screening large libraries of binding reagents or entities to identify combinations of binding reagents or entities that bind to the same target. In embodiments, the target is an EV, and the binding reagents or entities can target different surface markers (e.g., proteins) on the same EV. In embodiments, the target is a large macromolecule, for example, a single protein, and the binding reagents or entities can target different epitopes on the same protein. In embodiments, the target is a protein complex comprising one or more of the same protein monomers, and the binding reagents or entities can target the same epitope on different monomers.

In embodiments, the plurality of unique binding reagents or unique binding entities comprises at least ten unique binding reagents or unique binding entities. In embodiments, the plurality of unique binding reagents or unique binding entities comprises about 10 to about 1000 unique binding reagents or unique binding entities. In embodiments, the plurality of unique binding reagents or unique binding entities comprises about 10 to about 100 unique binding reagents or unique binding entities.

In embodiments, the multiplexed method is conducted in solution.

In embodiments, the methods described herein enable combinatorial screening and/or isolation of more than 10, more than 20, more than 30, more than 40, more than 50, more than 60, more than 70, more than 80, more than 90, more than 100, more than 500, or more than 1000 binding reagents or entities (e.g., antibodies) for EV surface markers in a single reaction. Thus, in embodiments, at least 103, 203, 303, 403, 503, 603, 703, 803, 903, 1003, 5003, or 10003 possible three-marker combinations can be screened in a single reaction. In embodiments, the multiple (e.g., three) binding reagents or entities also serve as a secondary tether, allowing selective removal of EVs lacking the combination of the three surface markers, thereby providing additional specificity.

In embodiments, the surface markers are identified by next-generation sequencing of the barcode oligonucleotide sequences associated with the binding reagents or entities specific for the surface markers. In embodiments, the same multiple (e.g., three) binding reagents (e.g., antibodies) identified by next-generation sequence are used for isolation of the EV, thereby simplifying reagent preparation and ensuring that the binding reagents (e.g., antibodies) behave similarly during both screening and isolation processes.

In embodiments, the EVs are isolated from monocytes. In embodiments, the EVs are isolated from B cells. In embodiments, the EVs are isolated from CD4+ T cells. In embodiments, the EVs are isolated from CD8+ T cells. In embodiments, the EVs are isolated from vascular endothelial cells.

### Iv. Examples

### Example 1—Proximity Ligation-Ligation Methods for Multimarker EV Detection/Selection

Constructs were designed for multimarker detection/selection of extracellular vesicles using a two-site proximity ligation-ligation (PLL) approach. This PLL approach has several advantages over a traditional proximity extension-ligation (PEL) approach, including: i) it is a single enzyme system requiring only ligase, as opposed to PEL which requires both ligase and polymerase; ii) it provides a higher efficiency conversion of bound antibodies into amplicon; and iii) shorter overlaps surrounding the ligation sites eliminate the need to perform antibody binding in low-salt buffers or to use oligonucleotide blockers.

An example of oligonucleotide conjugates used in an embodiment of a PLL method is show in FIG. 1A. A 5-prime conjugate comprises a surface marker binding agent, such as an antibody, conjugated to an oligonucleotide with sequences for a forward amplification primer, a first unique molecular identifier sequence (UMI 1), a first barcode and a first common overlap region (hybridization region). In this example, the 5′ end of the oligonucleotide is attached to the antibody. A splint conjugate comprises a surface marker binding agent, such as an antibody, conjugated to an oligonucleotide with sequences for a first common overlap region, a second barcode and a second common overlap region. In this example, the 3′ end of the oligonucleotide is attached to the antibody. A 3-prime conjugate comprises a surface marker binding agent, such as an antibody, conjugated to an oligonucleotide with sequences for a reverse amplification primer, a second unique molecular identifier sequence (UMI 2), a third barcode and a second common overlap region (hybridization region). In this example, the 3′ end of the oligonucleotide is attached to the antibody.

As shown in FIG. 1B (i), a bead attached to a capture entity (in this embodiment, a capture antibody) that binds an EV surface is used to capture the EV. As shown in FIG. 1B (ii), the 5-prime conjugate, splint conjugate and 3-prime conjugate are added to the EV captured on the bead, and the antibodies of each conjugate bind to their corresponding surface marker antigens on the EV. The labels 5′, 3′ and 3′ in FIG. 1B (ii) indicate the end of the nucleic acid that is conjugated to the antibody. As shown in FIG. 1B (iii), the first common overlap region on the 5-prime conjugate hybridizes with the first common overlap region on the splint conjugate and the second common overlap region on the splint conjugate hybridizes with the second common overlap region on the 3-prime construct. As shown in FIG. 1B (iv), an insert oligonucleotide that hybridizes to the unhybridized region of the splint construct is added and ligase is used to seal the construct into a single output oligonucleotide. Primers binding to the forward amplification primer and reverse amplification primer sites are added to amplify the single output oligonucleotide by PCR which is then sequenced as shown in FIG. 1B (v). Sequencing methods are preferably next-generation sequencing methods.

In embodiments sequence data is analyzed by comparing each barcode to a lookup table to identify the three surface marker binding agents that were conjugated to the three oligonucleotides composing each full length amplicon. Thus the sequencing data can be reduced to a list of three marker combinations or permutations. In embodiments, sequencing reads with identical UMIs will be binned and treated as a single read. In embodiments, the permutations of three markers may be analyzed. In embodiments the combinations of three markers may be analyzed. In embodiments, the number of sequencing reads with identical combinations will be determined for each unique combination or permutation to assess the frequency of each combination or permutation in the library. This frequency distribution may be used to assess the frequency of the corresponding three marker combinations on the surface marker displaying agents in the original sample. Three-marker combinations that are determined to be enriched on EVs or other SMDAs from a particular origin but not on EVs or SDMAs of other origins may be selected as specific indicators of that EV population. These three marker combinations may be used in assays to specifically detect or quantify that population or may be used to specifically enrich or purify that population from a mixed population of SDMAs or from a sample with other non-SDMA contaminants.

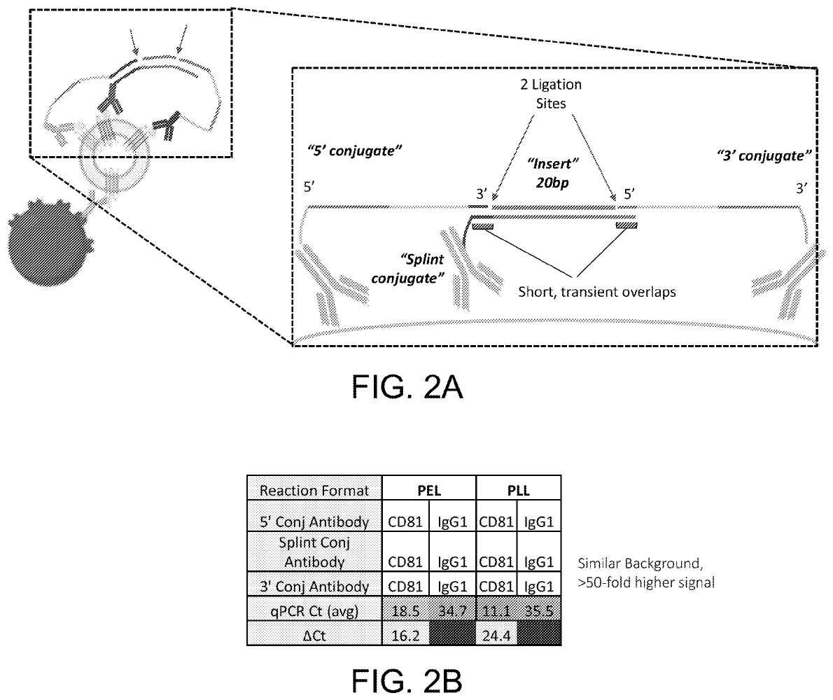

A close up schematic of the formation of the single output oligonucleotide is provided in FIG. 2A.

The PLL method was compared with the previously described PEL method with both CD81 and IgG1 as markers for the conjugates. In each case, beads were coated with anti CD63, CD81 and CD9 antibodies and these were used to capture CD81+EVs out of cell conditioned medium for approximately 2 hours. Beads were washed and exposed to various mixes of PLL conjugates with the insert oligos prehybridized or PEL conjugates for approximately 1 hour. After incubation with the conjugates, the beads were washed to remove unbound conjugates. The beads were transferred to either a ligation solution with T4 ligase in an appropriate buffer for PLL, or a mix of T4 ligase and T4 polymerase for PEL. This was followed by proteinase K digestion to release the full length DNA amplicons from the beads. A portion of each supernatant containing the release oligonucleotides was transferred to a qPCR reaction. Quantitative PCR was performed on the amplicon obtained from each method and the threshold cycle (Ct) was determined for each marker and method. As shown in FIG. 2B, while the PLL method had a similar background as determined by the Ct of the reaction using three negative control conjugates (denoted IgG1 in the figure), it provided a greater than 50-fold higher signal than the PEL method for the positive control condition using three CD81 conjugates. This indicates that the PLL reaction is more efficient in forming full length amplicons and should thus be more sensitive for detecting low abundance SMDAs.

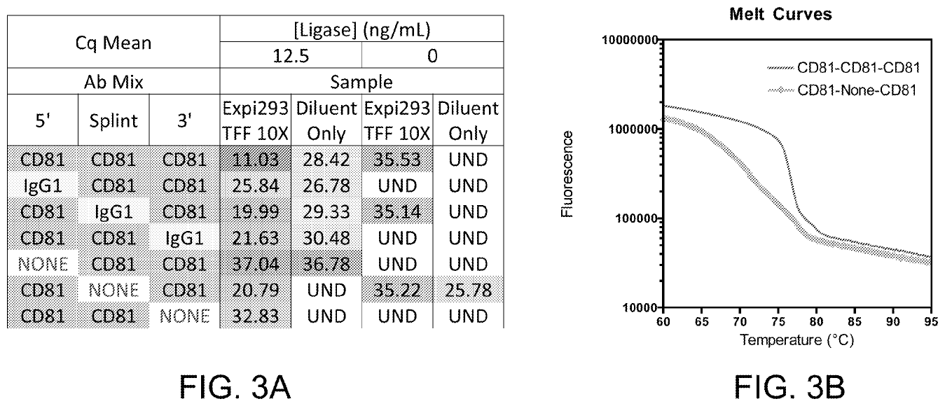

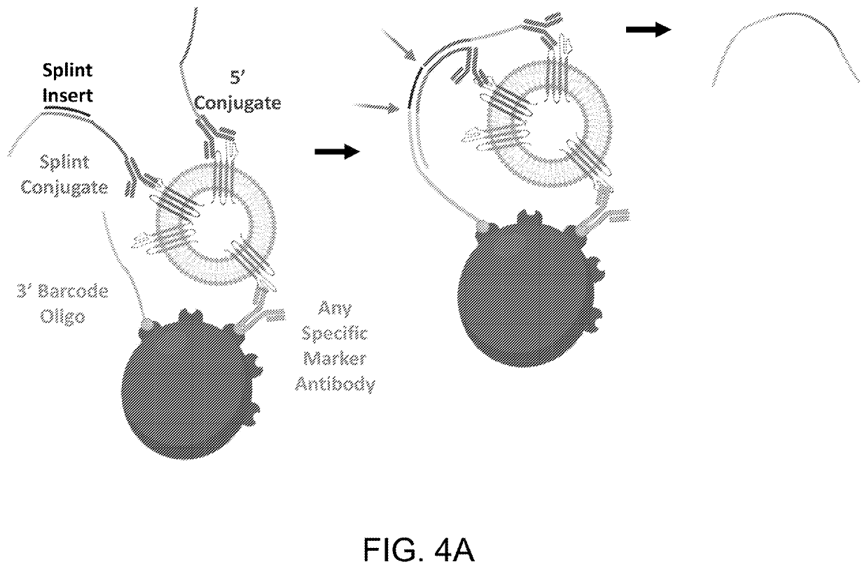

Further experiments were performed to assess the specificity of the PLL method. Results are summarized in FIG. 3A. As shown in the figure, a mixture of conjugates targeting either CD81 or IgG1 and no-conjugate controls were used in an experiment similar to that shown in FIG. 2B. If ligase was not added to the mixture, no signal was obtained. If EVs were not added, the change in Ct (ΔCt) observed was greater than 15 compared to samples containing EVs, representing a low background. When an IgG1 binding molecule was used in either the 5-prime or 3-prime construct, the ΔCt observed was greater than 10, consistent with the known non-specific binding level of the control antibody. And when no 5-prime or 3-prime construct was used, the ΔCt observed was greater than 20. As shown in FIG. 3A, use of an IgG1 splint construct or no splint construct caused a higher than expected signal. However, subsequent melting curve (FIG. 3B) and next-generation sequencing (NGS) analysis showed that this signal results from a 20-nt shorter product that is the result of splint-independent ligation of the 5-prime and 3-prime constructs. The truncated products formed from this splint-independent ligation can be easily rejected using NGS.

These results demonstrate the PLL assay can provide more reliable and sensitive signal than that obtainable with the PEL method.

### Example 2—Tetraspanin-Independent Proximity Ligation-Ligation

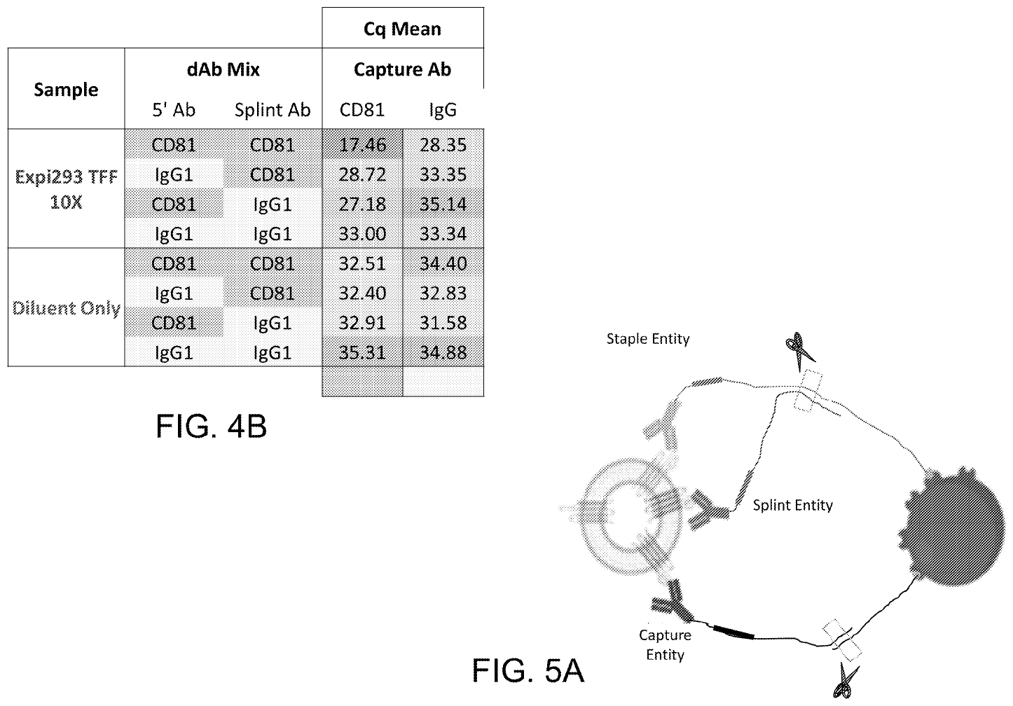

While tetraspanins are quite common EV surface markers, the inventors wished to develop a tetraspanin-independent assay that could be used with tetraspanin-negative EVs or other surface marker displaying agents. As shown schematically in FIG. 4A, this was achieved by attaching the 3-prime barcode oligonucleotide (an equivalent to the oligonucleotide on the 3-prime conjugate in Example 1 directly to the bead along with the capture reagent antibody. In embodiments, one, two, or more capture reagents can be provided, each capture reagent comprises a unique antibody and a specific 3-prime barcode oligonucleotide. The antibody of each capture reagent is capable of binding to a corresponding surface marker on the EV, and the antibody is linked to the specific 3-primer barcode oligonucleotide. In order to preserve the relationship between the capture reagent and it's corresponding barcoded oligonucleotide, a set of clonal beads was prepared for each capture reagent with the corresponding oligonucleotide, then the beads were mixed before performing the assay. As a simple demonstration we prepared two bead types, one with CD81 capture and the other with a non-specific control antibody. While we chose CD81 as an exemplary capture antibody target, any antibody suitable surface marker/capture antibody pair could be chosen, and many clonal bead types may be combined into one mix. The bead mixture was used to capture EVs from a cell conditioned media sample, followed by washing to remove unbound EVs and other contaminants. Various mixes of 5′ conjugates and splint conjugates were used to label the captured EVs, followed by washing and ligase treatment, as in Example 1. As shown in FIG. 4A, the PLL reaction occurs similarly to that described in Example 1 when surface markers for both the 5-prime conjugate and splint conjugate are present. Both the 5-prime conjugate and the splint conjugate bind to the captured EV. As the 3-prime barcode oligonucleotide is already present on the bead, the presence of the insert oligonucleotide (splint insert) allows the sequences to be ligated together to form the single output oligonucleotide which can be amplified and sequenced as described in Example 1. The resulting amplicons were released by proteinase K treatment and quantified by qPCR. The results are summarized in FIG. 6B. When CD81 capture beads are used along with CD81 5′ and splint conjugates (with splint insert prehybridized), the assay produces a cycle threshold of ˜17.5. Substitution of the IgG1 control antibody in to any of the three positions produces an increase in cycle threshold of approximately 10 cycles or more indicating that specific binding is required in each position to produce a strong positive signal. This is consistent with the results of Example 1. This assay can be used in either simultaneous or sequential format, with the components added all at once, including beads, sample and detectors or sequentially with the sample added to beads first, followed by the detection conjugates.

In order to determine additional antibodies that could be used as capture antibodies, approximately 200 antibodies for 75 different surface marker targets were screened. Desirable properties of the antibodies to be used were: i) a high affinity (Kd<1 nM); ii) that they bind the available epitope on an intact EV surface in human samples; and iii) that the antibody be available from a reputable supplier in a compatible formulation. After the analysis (data not shown), suitable antibodies for the 69 EV surface markers shown in Table 1 were selected.

TABLE 1

| CD2 | CD15 (SSEA-1) | CD36 (GPIV) | CD61 (GP3A) | CD81 | CD183 (CXCR3) | CX3CR1 |
|---|---|---|---|---|---|---|
| CD3 | CD16 (FcγRIII) | CD38 | CD62 (P- | CD95 (FAS) | CD204 (MSR1) | ITGB5 |
| | | | Selectin) | | | |
| CD4 | CD18 (ITGB2) | CD40 | CD62e (E- | CD105 (Endoglin) | CD223 (LAG-3) | HLA-A/B/C |
| | | | Selectin) | | | |
| CD5 | CD25(IL-2Ra) | CD41 (GP2B) | CD62L (L- | CD106 (VCAM-1) | CD309 | HLA- |
| | | | Selectin | | (VEGFR2) | DR/DP/DQ |
| CD8 | CD26(DPPIV) | CD42b(GP1B) | CD63 | CD127 (IL-7Ra) | CD324 (E- | ESAM |
| | | | | | Cadherin) | |
| CD9 | CD28 | CD42a (GP9) | CD64 (FcγRI) | CD141 | CD325 (N- | EGFR |
| | | | | (Thrombomodulin) | Cadherin) | |
| CD10 (NEP) | CD29 (ITGB1) | CD44 (HCAM) | CD66a | CD144 (VE-Cadherin) | CD326 (EpCAM) | FAPa |
| | | | (CEACAM1) | | | |
| CD11b | CD31 (PECAM- | CD45 (LCA) | CD66e | CD146 (MCAM) | CD340 (ERBB2) | FLT- |
| (ITGAM) | 1) | | (CEACAM5) | | | 1(VEGFR1) |
| CD13(AAP) | CD32b (FcγRII) | CD50 (ICAM3) | CD68 | CD163 | EphA2 | GLUT1 |
| | | | (LAMP4) | | | (SLC2A1) |
| CD14 | CD33 (Siglec-3) | CD54 (ICAM- | CD73 (NT5E) | CD166(ALCAM) | CD202B (TIE2) | |
| | | 1) | | | | |

Antibodies to any of the surface markers shown in Table 1 can be used as a capture antibody or as the antibody portion of any of the constructs described in Examples 1 and 2.

### Example 3—Improved Constructs for Isolation of Multimarker EVs

The inventors have also developed improved constructs and methods for multimarker isolation of EVs or other surface marker displaying agents (SMDAs). The methods of the disclosure involve one or more of the following advancements as depicted in FIGS. 5A and 5B:
A. Capture antibodies are attached to the surface by employing one or more oligonucleotide linkers with multiple uracil sites incorporated therein, thereby enabling release of the capture antibodies and captured EVs from the surface using a UDG1 (a base-excision enzyme). The UDG1 removes the uracils thus eliminating the hydrogen bonding and allowing the linker strands to dissociate. This method produced >95% release of captured EVs, similar to the previously used stringent wash, but allowed completely orthogonal release of the first and second linkages improving the reliability of the two or three-marker (EV stapling) selection process.
B. Highly specific endonuclease restriction sites (e.g, EcoRI site) are engineered into a selected region of one or more of the oligonucleotides forming the additional linkages (“staples”) that connect an EV or other SMDA to a surface, thereby enabling final EV release from the surface (e.g., a bead or plate) via restriction enzyme rather than a nonspecific nuclease like DNAse I. The restriction site may be positioned close to a ligation site that is used to form the second linkage (“staple”) between the captured EV and the surface. In this configuration, since the restriction enzyme only cleaves a specific site on the double stranded DNA, it will not release captured EVs in the absence of all required oligo-antibody conjugates (i.e., the splint and staple antibody conjugates as depicted in FIG. 5B), providing additional specificity, relative to a non-specific nuclease such as DNAse I.
C. Specific “target” sequences are incorporated into each oligonucleotide linkage that connects an EV or other SMDA to a surface through EV or SMDA surface markers. These can be incorporated into both the capture antibody linker and each of the one or more antibody conjugates used to form the second linkage (“staple”) and are located on the antibody-side of the restriction site or UDG cleavage site. These target sequences remain intact following EV release and can be hybridized to complementary dye-conjugated in situ probes (complementary to the “target” sequence) such that the EV can be fluorescently labeled for multi-color co-localization microscopy/FISH microscopy (i.e., a different fluorescent label is attached to each probe directed to a different target sequence). Accordingly, EVs can be stained after stapling for microscopy. Alternately, one or more of the target sequences can be hybridized to a complementary probe that is biotinylated for subsequent EV pulldown or recapture onto a streptavidin surface for additional analyses including ECL assays.

These improved constructs can be used in EV isolation, detection and sampling methods involving stapling linkages to the surface, for example, methods described herein and in US Patent Publication 2021/0382043, which is incorporated by reference in its entirety herein.

### Example 4—EV Assays of COVID Patients

Assays previously developed for intact EVs with putative CNS markers were applied to characterize the EVs in cerebrospinal fluid collected from patients with COVID, including those with and without neurological symptoms as well as non-COVID controls. These samples were previously tested, and it was shown that viral antigen was detectable in the CSF and correlated with immune activation. Neurosymptomatic COVID patients had increased proinflammatory cytokines and elevated markers of neuro-axonal injury, not attributable to the severity of COVID-19. To follow up on that testing, it was evaluated whether a distinct EV profile was observable in COVID patients relative to controls or in Neuro-COVID patients relative to non-neuro-COVID. A panel of 45 EV surface markers was selected that included proteins expected to be specific for neurons, astrocytes, microglia and oligodendrocytes. Elevated levels of EVs were observed with 15 surface markers in COVID patients relative to non-COVID controls shown in Table 2. It was observed that this list was highly enriched for microglia specific surface markers. The effect size was small but several of the results were highly significant, whether normalized by total EVs or not. When comparing NeuroCOVID to non-NeuroCOVID, the sample size was small but again, the elevated markers suggested microglia involvement (IBA1, MHC Class II and ITGB5). Several other markers were elevated but below the threshold for significance.

To see whether the use of multiple markers could improve the classification of these samples, all the putative microglia markers from the assay panel were selected (CX3CR1, MHC Class II, CD33, CD36, ITGB5, IBA1, CD14, CD40, CD64, Stabilin-1, GPNMB) and used in a principle component analysis. The first patient cohort was compared using a Wilcoxon test which produced a barely significant difference (P=0.046) between the Neuro and Non-neuro COVID groups. Eliminated variables were sequentially examined to identify the best list of microglia markers (ITGB5, CX3CR1, CD40, CD64, HLA-DR/DP/DQ, IBA1). This increased the significance (P=0.0093). This approach was tested with the astrocyte markers and with the neuron markers. In each case there was no combination of variables that produced significant discrimination between neuro and non-neuro COVID groups.

Lastly, the microglia EV populations were compared with previously measured cytokines and neuroinjury markers in the CSF samples. High correlations were observed between the proinflammatory cytokines IL6, TNFa, IL-1b, and IFNg and chemokines MCP-1 and IP-10 and microglia EV signals but not with other EV populations. COVID N-antigen only correlated with microglia marker IBA1. These analyses suggest that elevated microglia EVs, but not neuronal or astrocyte EVs are associated with neurological symptoms among COVID patients.

TABLE 2
Table 2: EV populations elevated in CSF from
COVID+ patients relative to non-COVID controls

| | COVID+ | COVID+ | COVID− | COVID− | Median | |
|---|---|---|---|---|---|---|
| Analyte | Median | IQR | Median | IQR | Ratio (+/−) | p-value |
| CD63 | 86,549 | 37,996 | 42,441 | 17,915 | 2.04 | 6.01E−12 |
| IBA1 | 1,136 | 649 | 582 | 215 | 1.95 | 5.16E−08 |
| GD2 | 1,358 | 566 | 712 | 311 | 1.91 | 5.09E−07 |
| CX3CR1 | 173,011 | 87,216 | 103,506 | 37,540 | 1.67 | 1.37E−05 |
| CD14 | 824 | 206 | 584 | 168 | 1.41 | 2.76E−03 |
| CD9 | 314,925 | 113,088 | 228,646 | 69,950 | 1.38 | 3.88E−06 |
| CD81 | 465,688 | 132,300 | 342,635 | 95,574 | 1.36 | 7.14E−07 |
| MAG | 7,943 | 4,789 | 6,066 | 2,462 | 1.31 | 5.28E−05 |
| CD24 | 852 | 428 | 659 | 386 | 1.29 | 1.74E−02 |
| CD32b | 519 | 214 | 414 | 39 | 1.25 | 3.77E−05 |
| CD40 | 761 | 337 | 616 | 216 | 1.24 | 3.69E−04 |
| CD307b | 902 | 359 | 773 | 363 | 1.17 | 3.20E−02 |
| ITGB5 | 625 | 197 | 543 | 82 | 1.15 | 1.66E−03 |
| NG2 | 1,081 | 427 | 950 | 349 | 1.14 | 1.46E−02 |
| GPNMB | 1,401 | 644 | 1,254 | 682 | 1.12 | 2.96E−02 |

1. A method of detecting an extracellular vesicle (EV) of interest in a sample, comprising:
a. contacting the sample with:
(i) a capture reagent bound to a surface;
(ii) a first binding reagent that binds a first surface marker of the EV, wherein the first binding reagent comprises a first detection sequence that comprises a first hybridization sequence, and a first amplification primer site;
(iii) a second binding reagent that binds a second surface marker of the EV, wherein the second binding reagent comprises a second detection sequence that comprises a second hybridization sequence, a third hybridization sequence, and a fourth hybridization sequence;
(iv) a third binding reagent comprising a third detection sequence, wherein the third detection sequence comprises a fifth hybridization sequence, and a second amplification primer site; and
(v) an oligonucleotide insert comprising an oligonucleotide insert sequence;
wherein the first hybridization sequence and the second hybridization sequence are complementary;
wherein the fourth hybridization sequence and the fifth hybridization sequence are complementary; and
wherein the third hybridization sequence is complementary to the oligonucleotide insert sequence;
b. forming a single output oligonucleotide comprising:
(i) ligating the hybridized first detection sequence, to the hybridized oligonucleotide insert; and
(ii) ligating the hybridized oligonucleotide insert to the third detection sequence; and
c. amplifying the single output oligonucleotide using a first primer that hybridizes to the first amplification primer site and a second primer that hybridizes to the second amplification primer site.

2. The method of claim 1, wherein the capture reagent is releasably bound to the surface.

3. (canceled)

4. (canceled)

5. The method of claim 1, wherein the third binding reagent comprising the third detection sequence is bound to the same surface as the capture reagent.

6. The method of claim 1, wherein the third binding reagent binds to a third surface marker of the EV.

7. The method of claim 1, wherein the method employs multiple different capture reagents and multiple different first, second, and third binding reagents.

8. The method of claim 1, further comprising: (i) detecting the amplified single output oligonucleotide; (ii) sequencing the amplified single output oligonucleotide; or (iii) both (i) and (ii).

9. The method of claim 1, wherein each of the first, second and third binding reagents comprises an antibody or antigen binding fragment thereof, antigen, ligand, receptor, oligonucleotide, hapten, epitope, mimitope, lipid binding protein, carbohydrate binding protein, DNA aptamer or RNA aptamer.

10. The method of claim 1, wherein each hybridization sequence: (i) has a length of about 5-10 nucleotides; (ii) is the same length; or (iii) both (i) and (ii).

11. (canceled)

12. (canceled)

13. The method of claim 1, wherein: (i) the first detection sequence further comprises a first barcode sequence between the first hybridization sequence and the first amplification primer site; (ii) the third detection sequence further comprises a third barcode sequence between the fifth hybridization sequence and the second amplification primer site; or (iii) both (i) and (ii).

14. The method of claim 13, wherein: (i) the first barcode sequence of the first detection sequence is a unique molecular identifier; the third barcode sequence of the third detection sequence is a unique molecular identifier; or (iii) both (i) and (ii).

15. (canceled)

16. (canceled)

17. The method of claim 1, wherein: (i) the first detection sequence and third detection sequence have a length of from about 35 to about 55 nucleotides; (ii) the oligonucleotide insert has a length of about 10 to about 30 nucleotides; or (iii) both (i) and (ii).

18-25. (canceled)

26. The method of claim 1, wherein the capture reagent binds to a surface marker that is not common to EVs.

27. (canceled)

28. A method of determining surface markers of a surface marker displaying agent comprising contacting the surface marker displaying agent with a capture reagent bound to a surface, a plurality of unique binding reagents and an oligonucleotide insert,
wherein each unique binding reagent comprises a detection sequence comprising a unique barcode oligonucleotide sequence;
wherein when at least three unique binding reagents bind to three unique surface markers of the surface marker displaying agent, an output oligonucleotide is generated that comprises the barcode oligonucleotide sequences of each of the three unique binding reagents;
wherein the output oligonucleotide is capable of being sequenced to identify the three unique surface markers of the surface marker displaying agent; and further
wherein the plurality of binding reagents comprises:
a. a first binding reagent comprising a first detection sequence that comprises a first hybridization sequence, and a first amplification primer site;
b. a second binding reagent comprising a second detection sequence that comprises a second hybridization sequence, a third hybridization sequence, and a fourth hybridization sequence; and
c. a third binding reagent comprising a third detection sequence that comprises a fifth hybridization sequence, and a second amplification primer site,
wherein the first hybridization sequence and the second hybridization sequence are complementary;
wherein the fourth hybridization sequence and the fifth hybridization sequence are complementary;
wherein the third hybridization sequence is complementary to the oligonucleotide insert sequence; and
wherein generating the output oligonucleotide comprises ligating the hybridized first detection sequence, to the hybridized oligonucleotide insert and ligating the hybridized oligonucleotide insert to the third detection sequence.

29-58. (canceled)

59. A method of isolating extracellular vesicles (EVs) from a sample, comprising contacting a sample suspected of containing EVs with
a first oligonucleotide-conjugated capture entity;
a second oligonucleotide-conjugated splint entity;
a third oligonucleotide-conjugated staple entity; and
a surface,
wherein each oligonucleotide-conjugated entity is specific for a different EV surface marker;
wherein the first oligonucleotide-conjugated capture entity comprises a first oligonucleotide comprising a first Target nucleotide sequence, and a UDG1 labile linkage sequence; wherein the capture entity is conjugated to the first oligonucleotide so that the first Target nucleotide sequence is located between the capture entity and the UDG1 labile linkage sequence;
wherein the third oligonucleotide-conjugated staple entity comprises a third oligonucleotide comprising a third Target nucleotide sequence and a restriction enzyme cleavage site; wherein the staple entity is conjugated to the third oligonucleotide so that the third Target nucleotide sequence is located between the staple entity and the restriction enzyme cleavage site;
wherein the second oligonucleotide-conjugated splint entity comprises a second oligonucleotide comprising a second Target nucleotide sequence, a restriction enzyme cleavage site and an additional nucleic acid sequence, wherein the splint entity is conjugated to the second oligonucleotide so that the second Target nucleotide sequence is located between the splint entity and the restriction enzyme cleavage site and the additional nucleic acid sequence is located on the side of the second Target nucleotide sequence opposite the side that is conjugated to the splint entity;
wherein complementary DNA sequences on the second oligonucleotide-conjugated splint entity and the third oligonucleotide-conjugated staple entity hybridize to form a double-stranded DNA restriction site; and
wherein the surface has two capture oligonucleotides immobilized thereon, wherein
the first capture oligonucleotide comprises a sequence that is complementary to the additional nucleic acid sequence of the second oligonucleotide of the second oligonucleotide-conjugated splint entity, and is capable of ligating to the end of the third oligonucleotide away from the third oligonucleotide-conjugated staple entity; and
the second capture oligonucleotide comprises a sequence that is complementary to the UDG1 labile linkage sequence of the first oligonucleotide on the first oligonucleotide-conjugated capture entity.

60. A construct for isolation of a multimarker extracellular vesicle (EV) from a sample, comprising:
a first oligonucleotide-conjugated capture entity;
a second oligonucleotide-conjugated splint entity;
a third oligonucleotide-conjugated staple entity; and
a surface,
wherein each oligonucleotide-conjugated entity is specific for a different EV surface marker;
wherein the first oligonucleotide-conjugated capture entity comprises a first oligonucleotide comprising a first Target nucleotide sequence, and a UDG1 labile linkage sequence; wherein the capture entity is conjugated to the first oligonucleotide so that the first Target nucleotide sequence is located between the capture entity and the UDG1 labile linkage sequence;
wherein the third oligonucleotide-conjugated staple entity comprises a third oligonucleotide comprising a third Target nucleotide sequence and a restriction enzyme cleavage site; wherein the staple entity is conjugated to the third oligonucleotide so that the third Target nucleotide sequence is located between the staple entity and the restriction enzyme cleavage site;
wherein the second oligonucleotide-conjugated splint entity comprises a second oligonucleotide comprising a second Target nucleotide sequence, a restriction enzyme cleavage site, and an additional nucleic acid sequence, wherein the splint entity is conjugated to the second oligonucleotide so that the second Target nucleotide sequence is located between the splint entity and the restriction enzyme cleavage site and the additional nucleic acid sequence is located on the side of the second Target nucleotide sequence opposite the side that is conjugated to the splint entity;
wherein complementary DNA sequences on the second oligonucleotide-conjugated splint entity and the third oligonucleotide-conjugated staple entity hybridize to form a double-stranded DNA restriction site; and
wherein the surface has two capture oligonucleotides immobilized thereon, wherein
the first capture oligonucleotide comprises a sequence that is complementary to the additional nucleic acid sequence of the second oligonucleotide of the second oligonucleotide-conjugated splint entity, and is capable of ligating to the end of the third oligonucleotide away from the third oligonucleotide-conjugated staple entity; and
the second capture oligonucleotide comprises a sequence that is complementary to the UDG1 labile linkage sequence of the first oligonucleotide on the first oligonucleotide-conjugated capture entity.

61. The method of claim 59, wherein the capture entity, splint entity and staple entity are each independently selected from an antibody or antigen binding fragment thereof, antigen, ligand, receptor, oligonucleotide, hapten, epitope, mimitope, lipid binding protein, carbohydrate binding protein, DNA aptamer or RNA aptamer.

62. (canceled)

63. The method of claim 59, wherein: (i) the capture entity of the first oligonucleotide-conjugated capture entity is conjugated to the 5′ end of the first oligonucleotide; (ii) the splint entity of the second oligonucleotide-conjugated splint entity is conjugated to the 3′ end of the second oligonucleotide; (iii) the staple entity of the third oligonucleotide-conjugated staple entity is conjugated to the 5′ end of the third oligonucleotide; or (iv) any combination of (i) to (iii).

64. (canceled)

65. (canceled)

66. The method of claim 59, wherein the first Target sequence, second Target sequence and third Target sequence have a length from about: (i) 15 to about 25 nucleotides; or (ii) 20 to about 30 nucleotides.

67. (canceled)

68. The method of claim 59, wherein the restriction enzyme cleavage site is a EcoRI, EcoRII, BamHI, HindIII, TaqI, NotI, HinFI, Sau3AI, PvuII, SmaI, HaeIII, HgaI, AluI, EcoRV, EcoP15I, KpnI, PstI, SacI, SalI, ScaI, SpeI, SphI, StuI or XbaI cleavage site.

69-83. (canceled)

84. A kit for detecting an EV in a sample comprising, in one or more vials, containers, or compartments:
(i) a capture reagent bound to a surface;
(ii) a first binding reagent that binds a first surface marker of the EV, wherein the first binding reagent comprises a first detection sequence that comprises a first hybridization sequence, and a first amplification primer site;
(iii) a second binding reagent that binds a second surface marker of the EV, wherein the second binding reagent comprises a second hybridization sequence, a third hybridization sequence, and a fourth hybridization sequence;
(iv) a third biding agent comprising a third detection sequence, wherein the third detection sequence comprises a fifth hybridization sequence, and a second amplification primer site; and
(v) an oligonucleotide insert comprising an oligonucleotide insert sequence,
wherein the first hybridization sequence and the second hybridization sequence are complementary;
wherein the fourth hybridization sequence and the fifth hybridization sequence are complementary; and
wherein the third hybridization sequence is complementary to the oligonucleotide insert sequence.

85-93. (canceled)

94. A kit for determining surface markers of a surface marker displaying agent (SMDA), for identifying SMDAs that harbor combinations of surface markers, for detecting populations of SMDAs having certain surface markers, and/or for detecting or quantifying multiple populations of SMDAs where each population has a specific set of surface markers, the kit comprising, in one or more vials, containers, or compartments:
a capture reagent,
at least three unique binding reagents and an oligonucleotide insert,
wherein each unique binding reagent comprises a detection sequence comprising a unique barcode oligonucleotide sequence;
wherein when at least three unique binding reagents bind to three unique surface markers of the surface marker displaying agent, an output oligonucleotide is generated that comprises the barcode oligonucleotide sequences of each of the three unique binding reagents;
wherein the output oligonucleotide is capable of being sequenced to identify the three unique surface markers of the surface marker displaying agent; and further
wherein the at least three unique binding reagents comprises:
a. a first binding reagent comprising a first detection sequence that comprises a first hybridization sequence, and a first amplification primer site;
b. a second binding reagent comprising a second detection sequence that comprises a second hybridization sequence, a third hybridization sequence, and a fourth hybridization sequence; and
c. a third binding reagent comprising a third detection sequence that comprises a fifth hybridization sequence, and a second amplification primer site,
wherein the first hybridization sequence and the second hybridization sequence are complementary;
wherein the fourth hybridization sequence and the fifth hybridization sequence are complementary; and
wherein the third hybridization sequence is complementary to the oligonucleotide insert sequence.

95-104. (canceled)

105. A kit for isolation of a multimarker extracellular vesicle (EV) from a sample, comprising, in one or more vials, containers, or compartments:
a first oligonucleotide-conjugated capture entity;
a second oligonucleotide-conjugated splint entity;
a third oligonucleotide-conjugated staple entity; and
a surface;
wherein each oligonucleotide-conjugated entity is specific for a different EV surface marker;
wherein the first oligonucleotide-conjugated capture entity comprises a first oligonucleotide comprising a first Target nucleotide sequence, and a UDG1 labile linkage sequence; wherein the capture entity is conjugated to the first oligonucleotide so that the first Target nucleotide sequence is located between the capture entity and the UDG1 labile linkage sequence;
wherein the third oligonucleotide-conjugated staple entity comprises a third oligonucleotide comprising a third Target nucleotide sequence and a restriction enzyme cleavage site, wherein the staple entity is conjugated to the third oligonucleotide so that the third Target nucleotide sequence is located between the staple entity and the restriction enzyme cleavage site;
wherein the second oligonucleotide-conjugated splint entity comprises a second oligonucleotide comprising a second Target nucleotide sequence, a restriction enzyme cleavage site, and an additional nucleic acid sequence, wherein the splint entity is conjugated to the second oligonucleotide so that the second Target nucleotide sequence is located between the splint entity and the restriction enzyme cleavage site and the additional nucleic acid sequence is located at the end of the second oligonucleotide that is not conjugated to the splint entity;
wherein complementary DNA sequences on the second oligonucleotide-conjugated splint entity and the third oligonucleotide-conjugated staple entity hybridize to form a double-stranded DNA restriction site; and
wherein the surface has two capture oligonucleotides immobilized thereon, wherein
the first capture oligonucleotide comprises a sequence that is complementary to the additional nucleic acid sequence of the second oligonucleotide of the second oligonucleotide-conjugated splint entity, and is capable of ligating to the end of the third oligonucleotide away from the third oligonucleotide-conjugated staple entity; and
the second capture oligonucleotide comprises a sequence that is complementary to the UDG1 labile linkage sequence of the first oligonucleotide on the first oligonucleotide-conjugated capture entity.

106-111. (canceled)


SEQUENCE LISTING (4 sequences)

SEQ ID NO: 1 - DNA, 62 bp, synthetic construct
  misc_feature 1..62: note = Synthetic
  source 1..62: mol_type = other DNA
aaaaaaaaaagtttatcgggcgtggtgctcgcatagaattcttgatgacgatgttggaggta

SEQ ID NO: 2 - DNA, 39 bp, synthetic construct
  misc_feature 1..39: note = Synthetic
  source 1..39: mol_type = other DNA
gtcatcaagaattcggtcttgacaaacgtgtgcttgtac

SEQ ID NO: 3 - DNA, 58 bp, synthetic construct
  misc_feature 1..58: note = Synthetic
  modified_base 27: mod_base = OTHER; note = uracil
  modified_base 30: mod_base = OTHER; note = uracil
  modified_base 33: mod_base = OTHER; note = uracil
  modified_base 37: mod_base = OTHER; note = uracil
  modified_base 42: mod_base = OTHER; note = uracil
  modified_base 46: mod_base = OTHER; note = uracil
  modified_base 50: mod_base = OTHER; note = uracil
  modified_base 54: mod_base = OTHER; note = uracil
  source 1..58: mol_type = other DNA
tatgaggacgaatctcccgcttataatgctaatacatacgatcagtcagtgcatcaca

SEQ ID NO: 4 - RNA, 33 nt, synthetic construct
  misc_feature 1..33: note = Synthetic
  source 1..33: mol_type = other RNA
aaaaaaaaaatgtgatgcactgactgatcgtat